(19) United States
(12) Patent Application Publication
(10) Pub. No.: US 2006/0204960 A1
(43) Pub. Date: Sep. 14, 2006

(54) Method for diagnosing diffuse-type gastric cancers

(75) Inventors: Yusuke Nakamura, Kanagawa (JP); Yoichi Furukawa, Kanagawa (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Oncotherapy Science, Inc., Kanagawa (JP)

(21) Appl. No.: 10/533,026

(22) PCT Filed: Sep. 19, 2003
(86) PCT No.: PCT/JP03/11975

§ 371 Date: Apr. 3, 2006

Related U.S. Application Data
(60) Provisional application No. 60/421,193, filed on Oct. 25, 2002.

(51) Int. Cl. A61K 48/00; C12Q 1/68; C07H 21/04; C12M 1/34

(52) U.S. Cl. 435/6; 435/287.2; 536/24.3; 514/44

(57) ABSTRACT

Objective methods for detecting and diagnosing diffuse-type gastric cancer (DGC) are described herein. In one embodiment, the diagnostic method involves the determining an expression level of DGC-associated gene that discriminate between DGC and normal cell. The present invention further provides methods of screening for therapeutic agents useful in the treatment of DGC, methods of treating DGC and method of vaccinating a subject against DGC.

### PRIORITY INFORMATION

This application claims priority to U.S. Provisional Application Ser. No. 60/421,193, filed Oct. 25, 2002.

### FIELD OF THE INVENTION

The invention relates to methods of diagnosing diffuse-type gastric cancers

### BACKGROUND OF THE INVENTION

Gastric cancer is the second leading cause of cancer death in the world (1). Surgery is still the mainstay in terms of treatment, because chemotherapy remains unsatisfactory. Gastric cancers at an early stage can be cured by surgical resection, but prognosis of advanced gastric cancers remains very poor.

Histological studies have classified gastric carcinomas into two distinct groups, the intestinal (or differentiated) type and the diffuse (or undifferentiated) type (2), having different features with regard to epidemiology, etiology, pathogenesis and biological behavior. The intestinal type occurs more commonly in elderly people and has better prognosis, but diffuse-type gastric cancer (DGC) is seen in relatively younger individuals without preference for either sex and displays a more invasive phenotype with a serious clinical course. Intestinal-type gastric cancer is presumed to result from atrophic gastritis, followed by progression to intestinal metaplasia and/or dysplasia (3), but the precursor lesion of the diffuse-type tumor is not known.

cDNA microarray technologies have enabled to obtain comprehensive profiles of gene expression in normal and malignant cells, and compare the gene expression in malignant and corresponding normal cells (Okabe et al., Cancer Res 61:2129-37 (2001); Kitahara et al., Cancer Res 61: 3544-9 (2001); Lin et al., Oncogene 21:4120-8 (2002); Hasegawa et al., Cancer Res 62:7012-7 (2002)). This approach enables to disclose the complex nature of cancer cells, and helps to understand the mechanism of carcinogenesis. Identification of genes that are deregulated in tumors can lead to more precise and accurate diagnosis of individual cancers, and to develop novel therapeutic targets (Bienz and Clevers, Cell 103:311-20 (2000)). To disclose mechanisms underlying tumors from a genome-wide point of view, and discover target molecules for diagnosis and development of novel therapeutic drugs, the present inventors have been analyzing the expression profiles of tumor cells using a cDNA microarray of 23040 genes (Okabe et al., Cancer Res 61:2129-37 (2001); Kitahara et al., Cancer Res 61:3544-9 (2001); Lin et al., Oncogene 21:4120-8 (2002); Hasegawa et al., Cancer Res 62:7012-7 (2002)).

Studies designed to reveal mechanisms of carcinogenesis have already facilitated identification of molecular targets for anti-tumor agents. For example, inhibitors of farnexyltransferase (FTIs) which were originally developed to inhibit the growth-signaling pathway related to Ras, whose activation depends on posttranslational farnesylation, has been effective in treating Ras-dependent tumors in animal models (He et al., Cell 99:335-45 (1999)). Clinical trials on human using a combination or anti-cancer drugs and anti-HER2 monoclonal antibody, trastuzumab, have been conducted to antagonize the proto-oncogene receptor HER2/neu; and have been achieving improved clinical response and overall survival of breast-cancer patients (Lin et al., Cancer Res 61:6345-9 (2001)). A tyrosine kinase inhibitor, STI-571, which selectively inactivates bcr-abl fusion proteins, has been developed to treat chronic myelogenous leukemias wherein constitutive activation of bcr-abl tyrosine kinase plays a crucial role in the transformation of leukocytes. Agents of these kinds are designed to suppress oncogenic activity of specific gene products (Fujita et al., Cancer Res 61:7722-6 (2001)). Therefore, gene products commonly up-regulated in cancerous cells may serve as potential targets for developing novel anti-cancer agents.

It has been demonstrated that CD8+ cytotoxic T lymphocytes (CTLs) recognize epitope peptides derived from tumor-associated antigens (TAAs) presented on MHC Class I molecule, and lyse tumor cells. Since the discovery of MAGE family as the first example of TAAs, many other TAAs have been discovered using immunological approaches (Boon, Int J Cancer 54: 177-80 (1993); Boon and van der Bruggen, J Exp Med 183: 725-9 (1996); van der Bruggen et al., Science 254: 1643-7 (1991); Brichard et al., J Exp Med 178: 489-95 (1993); Kawakami et al., J Exp Med 180: 347-52 (1994)). Some of the discovered TAAs are now in the stage of clinical development as targets of immunotherapy. TAAs discovered so far include MAGE (van der Bruggen et al., Science 254: 1643-7 (1991)), gp100 (Kawakami et al., J Exp Med 180: 347-52 (1994)), SART (Shichijo et al., J Exp Med 187: 277-88 (1998)), and NY-ESO-1 (Chen et al., Proc Natl Acad Sci USA 94: 1914-8 (1997)). On the other hand, gene products which had been demonstrated to be specifically overexpressed in tumor cells, have been shown to be recognized as targets inducing cellular immune responses. Such gene products include p53 (Umano et al., Brit J Cancer 84: 1052-7 (2001)), HER2/neu (Tanaka et al., Brit J Cancer 84: 94-9 (2001)), CEA (Nukaya et al., Int J Cancer 80: 92-7 (1999)), and so on.

In spite of significant progress in basic and clinical research concerning TAAs (Rosenbeg et al., Nature Med 4: 321-7 (1998); Mukherji et al., Proc Natl Acad Sci USA 92: 8078-82 (1995); Hu et al., Cancer Res 56: 2479-83 (1996)), only limited number of candidate TAAs for the treatment of adenocarcinomas, including colorectal cancer, are available. TAAs abundantly expressed in cancer cells, and at the same time which expression is restricted to cancer cells would be promising candidates as immunotherapeutic targets. Further, identification of new TAAs inducing potent and specific antitumor immune responses is expected to encourage clinical use of peptide vaccination strategy in various types of cancer (Boon and can der Bruggen, J Exp Med 183: 725-9 (1996); van der Bruggen et al., Science 254: 1643-7 (1991); Brichard et al., J Exp Med 178: 489-95 (1993); Kawakami et al., J Exp Med 180: 347-52 (1994); Shichijo et al., J Exp Med 187: 277-88 (1998); Chen et al., Proc Natl Acad Sci USA 94: 1914-8 (1997); Harris, J Natl Cancer Inst 88: 1442-5 (1996); Butterfield et al., Cancer Res 59: 3134-42 (1999); Vissers et al., Cancer Res 59: 5554-9 (1999); van der Burg et al., J Immunol 156: 3308-14 (1996); Tanaka et al., Cancer Res 57: 4465-8 (1997); Fujie et al., Int J Cancer 80: 169-72 (1999); Kikuchi et al., Int J Cancer 81: 459-66 (1999); Oiso et al., Int J Cancer 81: 387-94 (1999)).

It has been repeatedly reported that peptide-stimulated peripheral blood mononuclear cells (PBMCs) from certain healthy donors produce significant levels of IFN-γ in response to the peptide, but rarely exert cytotoxicity against tumor cells in an HLA-A24 or -A0201 restricted manner in ⁵¹Cr-release assays (Kawano et al., Cance Res 60: 3550-8 (2000); Nishizaka et al., Cancer Res 60: 4830-7 (2000); Tamura et al., Jpn J Cancer Res 92: 762-7 (2001)). However, both of HLA-A24 and HLA-A0201 are one of the popular HLA alleles in Japanese, as well as Caucasian (Date et al., Tissue Antigens 47: 93-101 (1996); Kondo et al., J Immunol 155: 4307-12 (1995); Kubo et al., J Immunol 152: 3913-24 (1994); Imanishi et al., Proceeding of the eleventh International Hictocompatibility Workshop and Conference Oxford University Press, Oxford, 1065 (1992); Williams et al., Tissue Antigen 49: 129 (1997)). Thus, antigenic peptides of carcinomas presented by these HLAs may be especially useful for the treatment of carcinomas among Japanese and Caucasian. Further, it is known that the induction of low-affinity CTL in vitro usually results from the use of peptide at a high concentration, generating a high level of specific peptide/MHC complexes on antigen presenting cells (APCs), which will effectively activate these CTL (Alexander-Miller et al., Proc Natl Acad Sci USA 93: 4102-7 (1996)).

### SUMMARY OF THE INVENTION

The invention is based the discovery of a pattern of gene expression correlated with DGC, e.g., adenocarcinoma. The genes that are differentially expressed in DGC are collectively referred to herein as “DGC nucleic acids” or “DGC polynucleotides” and the corresponding encoded polypeptides are referred to as “DGC polypeptides” or “DGC proteins.”

Accordingly, the invention features a method of diagnosing or determining a predisposition to developing DGC in a subject by determining an expression level of a DGC-associated gene in a patient derived biological sample, such as tissue sample. By DGC-associated gene is meant a gene that is characterized by an expression level which differs in a cell obtained from a DGC cell compared to a normal cell. A normal cell is one obtained from gastric tissue known not to be cancerous. A DGC-associated gene includes e.g., one or more of DGC 1-463. An alteration, e.g., increase or decrease of the level of expression of the gene compared to a normal control level of the gene indicates that the subject suffers from or is at risk of developing DGC.

By normal control level is meant a level of gene expression detected in a normal, healthy individual or in a population of individuals known not to be suffering from DGC. A control level is a single expression pattern derived from a single reference population or from a plurality of expression patterns. For example, the control level can be a database of expression patterns from previously tested cells.

An increase in the level of DGC 1-136 detected in a test sample compared to a normal control level indicates the subject (from which the sample was obtained) suffers from or is at risk of developing DGC. In contrast, a decrease in the level of DGC 137-463 detected in a test sample compared to a normal control level indicates said subject suffers from or is at risk of developing DGC.

Alternatively, expression of a panel of DGC-associated genes in the sample is compared to a DGC reference level of the same panel of genes. By DGC reference level is meant the expression profile of the DGC-associated genes found in a population suffering from DGC.

Gene expression is increased or decreased 10%, 25%, 50% compared to a normal control level. Alternately, gene expression is increased or decreased 1, 2, 5 or more fold compared to a normal control level. Expression is determined by detecting hybridization, e.g., on an array, of a DGC-associated gene probe to a gene transcript or copy thereof of the patient-derived tissue sample.

The patient derived tissue sample is any tissue from a test subject, e.g., a patient known to or suspected of having DGC. For example, the tissue contains sputum, blood, serum, plasma, or a gastric cell (e.g., biopsy sample obtained from the stomach, small intestine, large intestine or lymph node tissue).

The invention also provides a DGC reference expression profile of a gene expression level of two or more of DGC 1-463. Alternatively, the invention provides a DGC reference expression profile of the levels of expression two or more of DGC 1-136 or DGC 137-463.

The invention further provides methods of identifying an agent that inhibits or enhances the expression or activity of a DGC-associated gene, e.g., DGC 1-463 by contacting a test cell expressing a DGC-associated gene with a test agent and determining the expression level of the DGC-associated gene. The test cell is a gastric cell such as a gastric mucosal cell or submucosal cell. A decrease of the level of DGC 1-136 in the presence of the tests agent compared to a control level (e.g., in the absence of the test agent) of the gene indicates that the test agent is an inhibitor of the DGC-associated gene and reduces a symptom of DGC. Alternatively, an increase of the level or activity of DGC 137-463 in the presence of the test agent compared to a normal control level or activity of the gene indicates that said test agent is an enhancer of expression or function of the DGC-associated gene and reduces a symptom of DGC.

The invention also provides a kit with a detection reagent which binds to two or more DGC nucleic acid sequences or which binds to a gene product encoded by the nucleic acid sequences. Also provided is an array of nucleic acids that binds to two or more DGC nucleic acids.

Therapeutic methods include a method of treating or preventing DGC in a subject by administering to the subject an antisense composition. The antisense composition reduces the expression of a specific target gene, e.g., the antisense composition contains a nucleotide, which is complementary to a sequence selected from the group consisting of DGC 1-136. Another method includes the steps of administering to a subject a short interfering RNA (siRNA) composition. The siRNA composition reduces the expression of a nucleic acid selected from the group consisting of DGC 1-136. In yet another method, treatment or prevention of DGC in a subject is carried out by administering to a subject a ribozyme composition. The nucleic acid-specific ribozyme composition reduces the expression of a nucleic acid selected from the group consisting of DGC 1-136. Other therapeutic methods include those in which a subject is administered a compound that increases the expression of DGC 137-463 or activity of a polypeptide encoded by DGC 137-463. Furthermore, DGC can be treated by administering a protein encoded by DGC 137-463. The protein may be directly administered to the patient or, alternatively, may be expressed in vivo subsequent to being introduced into the patient, for example, by administering an expression vector or host cell carrying the down-regulated marker gene of interest. Suitable mechanisms for in vivo expression of a gene of interest are known in the art.

The invention also includes vaccines and vaccination methods. For example, a method of treating or preventing DGC in a subject is carried out by administering to the subject a vaccine containing a polypeptide encoded by a nucleic acid selected from the group consisting of DGC 1-136 or an immunologically active fragment such a polypeptide. An immunologically active fragment is a polypeptide that is shorter in length than the full-length naturally-occurring protein and which induces an immune response. For example, an immunologically active fragment at least 8 residues in length and stimulates an immune cell such as a T cell or a B cell. Immune cell stimulation is measured by detecting cell proliferation, elaboration of cytokines (e.g., IL-2), or production of an antibody.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE FIGURES

FIG. 1 is a photograph a gene expression assay showing the level of expression of the five commonly up-regulated genes in the microarray data. Semi-quantitative RT-PCR experiment of the five genes was carried out using RNAs from eight DGCs and the corresponding non-cancerous mucosal tissues. T, cancer tissue; N, non-cancerous mucosa Expression of FDFT1 served as an internal control.

### DETAILED DESCRIPTION

The data described herein represents the first expression analysis of genome-wide genes in that type of cancer. Unlike other studies, e.g., one using an oligonucleotide array representing 6,800 genes to examine expression in scirrhous-type gastric-cancer cell lines and another using a cDNA array consisting of 1174 genes to analyze expression profiles in xenografts of human intestinal-type and diffuse-type gastric tumors, the present data provides a genome-wide expression profiles of DGC obtained from measuring the expression of over 23,000 genes in clinical samples.

Since DGC cells do not form large nests and infiltrate into the wall of stomach, laser-microbeam microdissection had the great advantage of separating cancer cells from interstitial tissues. This method of obtaining cells offers advantages over existing methods in that the percentage of contaminated cells of this method was significantly less than previous methods. Hence, the present data reflects expression profiles of highly pure population of diffuse-type tumor cells.

The methods allow early, sensitive, and reliable identification of individuals of diffuse-type gastric tumors. For example, tumors or a predisposition to developing tumors are detected prior to identification of overt clinical symptoms. Early detection is particularly important as this type of cancer is aggressive and affects a younger population. Intervention at a stage prior to the manifestation of overt clinical symptoms is important in reducing mortality from this cancer type. Another advantage of the present methods is that the data is objective, i.e., a measurable increase or decrease in gene expression, compared to a subjective (and therefore more error-prone) standard histological methods.

The present invention is based in part on the discovery of changes in expression patterns of multiple nucleic acid sequences in gastric mucosa tissue from primary gastric cancer tissue of patients with diffuse-type gastric adenocarcinoma compared to non-cancerous gastric control tissue. The differences in gene expression were identified by using a comprehensive cDNA microarray system and a laser-microbeam microdissection techinque.

DGC cells do not form large nests and infiltrate into the wall of stomach, thus laser-microbeam microdissection had the great advantage of separating cancer cells from interstitial tissues. The percentage of contaminated cells of this method was estimated less than 0.3%. Thus the expression profiles described herein represent a highly pure population of diffuse-type tumor cells.

cDNA microarray analysis was performed on over 20,000 genes and genes that were consistently and reliably over-expressed or suppressed among DGC patients were selected. 463 genes were found to be differentially expressed in more than 50% of the samples examined 136 genes were up-regulated and 327 were down-regulated.

The differentially expressed genes identified herein are used for diagnostic purposes and to develop gene targeted therapeutic approaches to inhibiting DGC.

The genes whose expression levels are modulated (i.e., increased or decreased) in DGC patients are summarized in Tables 1-2 and are collectively referred to herein as “DGC-associated genes”, “DGC nucleic acids” or “DGC polynucleotides” and the corresponding encoded polypeptides are referred to as “DGC polypeptides” or “DGC proteins.” Unless indicated otherwise, “DGC” is meant to refer to any of the sequences disclosed herein. (e.g., DGC 1-463). The genes have been previously described and are presented along with a database accession number.

By measuring expression of the various genes in a sample of cells, the presence of DGC is determined in a cell or population of cells. Similarly, by measuring the expression of these genes in response to various agents, and agents for treating DGC can be identified.

The invention involves determining (e.g., measuring) the expression of at least one, and up to all the DGC sequences listed in Tables 1-2. Preferably, one or more DGC-associated gene is measured in conjunction with other genes known to be associated with gastric cancers such as for example K-ras, CTNNB1 (β-catenin), c-erbB-2, K-sam, cyclinE, c-met p53, RB, APC, DCC and CDH1 (E-cadherin). Alternatively, the methods do not involve detecting the level of expression of one or more of the foregoing genes. Using sequence information provided by the GenBank® database entries for the known sequences the DGC-associated genes are detected and measured using techniques well known to one of ordinary skill in the art. For example, sequences within the sequence database entries corresponding to DGC sequences, are used to construct probes for detecting DGC RNA sequences in, e.g., northern blot hybridization analysis. Probes are preferably 10, 25, 50, 250 500, 1000, 2000 nucleotides in length and up to the full length reference sequence. As another example, the sequences can be used to construct primers for specifically amplifying the DGC sequences in, e.g, amplification-based detection methods such as reverse-transcription based polymerase chain reaction.

Expression level of one or more of the DGC sequences in the test cell population, e.g., a patient derived tissue sample is then compared to expression levels of the same sequence in a reference population. The reference cell population includes one or more cells for which the compared parameter is known, i.e., cancerous or non-cancerous.

Whether or not the gene expression level in the test cell population compared to the reference cell population reveals the presence of the measured parameter depends upon the composition of the reference cell population. For example, if the reference cell population is composed of non-cancerous cells, a similar gene expression level in the test cell population and reference cell population indicates the test cell population is non-cancerous. Conversely, if the reference cell population is made up of cancerous cells, a similar gene expression profile between the test cell population and the reference cell population that the test cell population includes cancerous cells.

The level of expression of a DGC nucleic acid or polypeptide in a test cell population is considered altered if its expression level varies from the reference cell population by more than 1.0, 1.5, 2.0, 5.0, 10.0 or more fold from the expression level of the corresponding DGC sequence in the reference cell population.

If desired, comparison of differentially expressed genes between a test cell population and a reference cell population can be done with respect to a control nucleic acid whose expression is independent of the parameter or condition being measured. For example, a control nucleic acid is one which is known not to differ depending on the cancerous or non-cancerous state of the cell. Expression levels of the control nucleic acid in the test and reference nucleic acid can be used to normalize signal levels in the compared populations. Control genes can be, e.g,. β-actin, glyceraldehyde 3-phosphate dehydrogenase or ribosomal protein P1.

The test cell population is compared to multiple reference cell populations. Each of the multiple reference populations may differ in the known parameter. Thus, a test cell population may be compared to a second reference cell population known to contain, e.g., DGC cells, as well as a second reference population known to contain, e.g., non-DGC cells (normal cells). The test cell is included in a tissue type or cell sample from a subject known to contain, or to be suspected of containing, DGC cells.

The test cell is obtained from a bodily tissue or a bodily fluid, e.g., biological fluid (such as blood, serum, feces or sputum). For example, the test cell is purified from a tissue. Preferably, the test cell population comprises a gastric cell. The gastric cell is from tissue known to be or suspected to be DGC.

Cells in the reference cell population are derived from a tissue type as similar to test cell. Alternatively, the control cell population is derived from a database of molecular information derived from cells for which the assayed parameter or condition is known.

The subject is preferably a mammal. The mammal can be, e.g., a human, nonhuman primate, mouse, rat, dog, cat, horse, or cow.

The expression of 1, 2, 3, 4, 5, 25, 35, 50, or 100 or more of the sequences represented by DGC 1-463 is determined and if desired, expression of these nucleic acid sequences can be determined along with other sequences whose level of expression is known to be altered according to one of the herein described parameters or conditions, e.g., DGC or non-DGC.

Expression of the genes disclosed herein is determined at the RNA level using any method known in the art For example, Northern hybridization analysis using probes which specifically recognize one or more of these sequences can be used to determine gene expression. Alternatively, expression is measured using reverse-transcription-based PCR assays, e.g., using primers specific for the differentially expressed sequences.

Expression is also determined at the protein level, i.e., by measuring the levels of polypeptides encoded by the gene products described herein, or biological activity thereof. Such methods are well known in the art and include, e.g., immunoassays based on antibodies to proteins encoded by the genes. The biological activity of the proteins encoded by the genes is also well known.

Diagnosing DGC

DGC is diagnosed by examining the expression of one or more DGC nucleic acid sequences from a test population of cells, (i.e., a patient derived biological sample). Preferably, the test cell population comprises a gastric cell, e.g., a cell obtained from the gastrointestinal system. Gene expression is also measured from blood, feces or other bodily fluids such as sputum. Other biological samples can be used for measuring the protein level. For example, the protein level in the blood, or serum derived from subject to be diagnosed can be measured by immunoassay or biological assay.

Expression of one or more of a DGC-associated gene, e.g., DGC 1-463 is determined in the test cell or biological sample and compared to the expression of the normal control level. By normal control level is meant the expression profile of the DGC-associated genes typically found in a population know not to be suffering from DGC. An increase or a decrease of the level of expression in the patient derived tissue sample of the DGC-associated genes compared to a normal control level indicates that the subject is suffering from or is at risk of developing DGC. For example, an increase in expression of DGC 1-136 in the test population compared to the normal control level indicates that the subject is suffering from or is at risk of developing DGC. Conversely, a decrease in expression of DGC 137-463 in the test population compared to the normal control level indicates that the subject is suffering from or is at risk of developing DGC.

When one or more of the DGC-associated genes are altered in the test population compared to the normal control level indicates that the subject suffers from or is at risk of developing DGC. For example, an alteration of 10%, 20%, 50%, 60%, 80%, 90% or more of the DGC-associated genes identified herein indicates a diagnosis of DGC.

Identifying Agents that Inhibit or Enhance DGC-Associated Gene Expression

An agent that inhibits the expression or activity of a DGC-associated gene is identified by contacting a test cell population expressing a DGC-associated up-regulated gene with a test agent and determining the expression level of the DGC-associated gene. A decrease in expression of a gastric cancer-associated gene such as DGC 1-136 compared to the control level indicates the agent is an inhibitor of a DGC-associated up-regulated gene and useful to inhibit DGC.

Alternatively, an agent that enhances the expression or activity of a DGC-associated down-regulated gene is identified by contacting a test cell population expressing a DGC-associated gene with a test agent and determining the expression level or activity of the DGC-associated down-regulated gene. An increase of expression or activity compared to a control level of the DGC-associated gene indicates that said test agent is an enhancer of the DGC-associated gene.

The test cell population is any cell expressing the DGC-associated genes. For example, the test cell population contains a gastric epithelial cell. For example, the test cell is immortalized cell line derived from a DGC cell. Alternatively, the test cell is a cell, which has been transfected with a PNC-associated gene or which has been transfected with a regulatory sequence (e.g. promoter sequence) from a PNC-associated gene operably linked to a reporter gene.

Assessing Efficacy of Treatment of DGC in a Subject

The differentially expressed DGC-associated genes identified herein also allow for the course of treatment of DGC to be monitored. In this method, a test cell population is provided from a subject undergoing treatment for DGC. If desired, test cell populations are obtained from the subject at various time points before, during, or after treatment Expression of one or more of the DGC-associated genes, in the cell population is then determined and compared to a reference cell population which includes cells whose DGC state is known. The reference cells have not been exposed to the treatment.

If the reference cell population contains no DGC cells, a similarity in expression between DGC-associated genes in the test cell population and the reference cell population indicates that the treatment is efficacious or conferring clinical benefit. However, a difference in expression between DGC sequences in the test population and this reference cell population indicates a less favorable clinical outcome or prognosis.

By “efficacious” is meant that the treatment leads to a reduction in expression of a pathologically up-regulated gene, increase in expression of a pathologically down-regulated gene or a decrease in size, prevalence, or metastatic potential of DGC in a subject. When treatment is applied prophylactically, “efficacious” means that the treatment retards or prevents DGC from forming. Assessment of the stage of DGC is made using standard clinical protocols.

Efficaciousness is determined in association with any known method for diagnosing or treating DGC. DGC is diagnosed for example, by identifying symptomatic anomalies, e.g., indigestion, difficult swallowing, anemia, vomiting blood, blood clots, blood in stool or fecal occult blood test, CT scan and gastroscopy.

Selecting a Therapeutic Agent for Treating DGC that is Appropriate for a Particular Individual

Differences in the genetic makeup of individuals can result in differences in their relative abilities to metabolize various drugs. An agent that is metabolized in a subject to act as an anti-DGC agent can manifest itself by inducing a change in gene expression pattern in the subject's cells from that characteristic of a cancerous state to a gene expression pattern characteristic of a non-cancerous state. Accordingly, the differentially expressed DGC-associated genes disclosed herein allow for a putative therapeutic or prophylactic anti-DGC agent to be tested in a test cell population from a selected subject in order to determine if the agent is a suitable anti-DGC agent in the subject.

To identify an anti-DGC agent, that is appropriate for a specific subject, a test cell population from the subject is exposed to a therapeutic agent, and the expression of one or more of DGC 1-463 genes is determined.

The test cell population contains a DGC cell expressing a DGC-associated gene. Preferably, the test cell is an epithelial cell. For example a test cell population is incubated in the presence of a candidate agent and the pattern of gene expression of the test sample is measured and compared to one or more reference profiles, e.g., a DGC reference expression profile or an non-DGC reference expression profile.

A decrease in expression of one or more of the sequences DGC 1-136 or an increase in expression of one or more of the sequences DGC 137-463 in a test cell population relative to a reference cell population containing DGC is indicative that the agent is therapeutic. The test agent can be any compound or composition. For example, the test agents are immunomodulatory agents, specific antisense nucleotide compounds which correspond to an aberrantly over-expressed DGC nucleic acid, polypeptide of agents that augment the expression of an aberrantly under-expressed DGC nucleic acid or polypeptide in the particular individual to be treated.

Screening Assays for Identifying Therapeutic Agents

The differentially expressed genes disclosed herein can also be used to identify candidate therapeutic agents for treating DGC. The method is based on screening a candidate therapeutic agent to determine if it converts an expression profile of DGC 1-463 sequences characteristic of a DGC state to a pattern indicative or more similar to that of a clinical state that is not associated with DGC.

In the method, a cell is exposed to a test agent or a combination of test agents (sequentially or consecutively) and the expression of one or more DGC 1-463 in the cell is measured. The expression profile of the DGC-associated genes in the test population is compared to expression level of the DGC-associated genes in a reference cell population that is not exposed to the test agent.

There is no limitation on the type of candidate agent in the screening of the present invention. The candidates of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997)Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Bio Techniques 13:412), or on beads (Lam (1991) Nature 354:82), chips (Fodor (1993) Nature 364:555), bacteria (U.S. Pat No. 5,223,409), spores (U.S. Pat. Nos. 5,571,698; 5,403,484; and 5,223,409), plasmids (Cull et al. (1992) Proc. Natl. Acad. Sci. USA 89:1865) or phage (Scott and Smith (1990) Science 249:386; Devlin (1990) Science 249:404; Cwirla et al. (1990) Proc. Natl. Acad. Sci. USA 87:6378; and Felici (1991) J. Mol. Biol. 222:301). (United States Patent Application 20020103480)

An agent effective in stimulating expression of underexpressed genes, or in suppressing expression of overexpressed genes is deemed to lead to a clinical benefit. Such compounds are further tested for the ability to prevent cancer cell growth.

In a further embodiment, the present invention provides methods for screening candidate agents which are potential targets in the treatment of DGC. As discussed in detail above, by controlling the expression levels or activities of marker genes, one can control the onset and progression of DGC. Thus, candidate agents, which are potential targets in the treatment of DGC, can be identified through screenings that use the expression levels and activities of marker genes as indices. In the context of the present invention, such screening may comprise, for example, the following steps:
a) contacting a test compound with a polypeptide encoded by a nucleic acid selected from the group consisting of DGC 1-463;
b) detecting the binding activity between the polypeptide and the test compound; and
c) selecting a compound that binds to the polypeptide

Alternatively, the screening method of the present invention may comprise the following steps:
a) contacting a candidate compound with a cell expressing one or more marker genes, wherein the one or more marker genes is selected from the group consisting of DGC 1-463; and
b) selecting a compound that reduces the expression level of one or more marker genes selected from the group consisting of DGC 1-136, or elevates the expression level of one or more marker genes selected from the group consisting of DGC 137-463.

Cells expressing a marker gene include, for example, cell lines established from DGC; such cells can be used for the above screening of the present invention.

Alternatively, the screening method of the present invention may comprise the following steps:
a) contacting a test compound with a polypeptide encoded by a nucleic acid selected from the group consisting of selected from the group consisting of DGC 1-463;
b) detecting the biological activity of the polypeptide of step (a); and
c) selecting a compound that suppresses the biological activity of the polypeptide encoded by a nucleic acid selected from the group consisting of DGC 1-136 in comparison with the biological activity detected in the absence of the test compound, or enhances the biological activity of the polypeptide encoded by a nucleic acid selected from the group consisting of DGC 137-463 in comparison with the biological activity detected in the absence of the test compound.

A protein required for the screening can be obtained as a recombinant protein using the nucleotide sequence of the marker gene. Based on the information of the marker gene, one skilled in the art can select any biological activity of the protein as an index for screening and a measurement method based on the selected biological activity.

Alternatively, the screening method of the present invention may comprise the following steps:
a) contacting a candidate compound with a cell into which a vector comprising the transcriptional regulatory region of one or more marker genes and a reporter gene that is expressed under the control of the transcriptional regulatory region has been introduced, wherein the one or more marker genes are selected from the group consisting of DGC 1-463
b) measuring the activity of said reporter gene; and
c) selecting a compound that reduces the expression level of said reporter gene when said marker gene is an up-regulated marker gene selected from the group consisting of DGC 1-136 or that enhances the expression level of said reporter gene when said marker gene is a down-regulated marker gene selected from the group consisting of DGC 137-463, as compared to a control.
   Suitable reporter genes and host cells are well known in the art. The reporter construct required for the screening can be prepared by using the transcriptional regulatory region of a marker gene. When the transcriptional regulatory region of a marker gene has been known to those skilled in the art, a reporter construct can be prepared by using the previous sequence information. When the transcriptional regulatory region of a marker gene remains unidentified, a nucleotide segment containing the transcriptional regulatory region can be isolated from a genome library based on the nucleotide sequence information of the marker gene.

The compound isolated by the screening is a candidate for drugs that inhibit the activity of the protein encoded by marker genes and can be applied to the treatment or prevention of DGC.

Moreover, compound in which a part of the structure of the compound inhibiting the activity of proteins encoded by marker genes is converted by addition, deletion and/or replacement are also included in the compounds obtainable by the screening method of the present invention.

When administrating the compound isolated by the method of the invention as a pharmaceutical for humans and other mammals, such as mice, rats, guinea-pigs, rabbits, cats, dogs, sheep, pigs, cattle, monkeys, baboons, and chimpanzees, the isolated compound can be directly administered or can be formulated into a dosage form using known pharmaceutical preparation methods. For example, according to the need, the drugs can be taken orally, as sugar-coated tablets, capsules, elixirs and microcapsules, or non-orally, in the form of injections of sterile solutions or suspensions with water or any other pharmaceutically acceptable liquid. For example, the compounds can be mixed with pharmaceutically acceptable carriers or media, specifically, sterilized water, physiological saline, plant-oils, emulsifiers, suspending agents, surfactants, stabilizers, flavoring agents, excipients, vehicles, preservatives, binders, and such, in a unit dose form required for generally accepted drug implementation. The amount of active ingredients in these preparations makes a suitable dosage within the indicated range acquirable.

Examples of additives that can be mixed to tablets and capsules are, binders such as gelatin, corn starch, tragacanth gum and arabic gum; excipients such as crystalline cellulose; swelling agents such as corn starch, gelatin and alginic acid; lubricants such as magnesium stearate; sweeteners such as sucrose, lactose or saccharin; and flavoring agents such as peppermint, Gaultheria adenothrix oil and cherry. When the unit-dose form is a capsule, a liquid carrier, such as an oil, can also be further included in the above ingredients. Sterile composites for injections can be formulated following normal drug implementations using vehicles such as distilled water used for injections.

Physiological saline, glucose, and other isotonic liquids including adjuvants, such as D-sorbitol, D-mannnose, D-mannitol, and sodium chloride, can be used as aqueous solutions for injections. These can be used in conjunction with suitable solubilizers, such as alcohol, specifically ethanol, polyalcohols such as propylene glycol and polyethylene glycol, non-ionic surfactants, such as Polysorbate 80™ and HCO-50.

Sesame oil or Soy-bean oil can be used as a oleaginous liquid and may be used in conjunction with benzyl benzoate or benzyl alcohol as a solubilizer and may be formulated with a buffer, such as phosphate buffer and sodium acetate buffer; a pain-killer, such as procaine hydrochloride; a stabilizer, such as benzyl alcohol and phenol; and an anti-oxidant. The prepared injection may be filled into a suitable ampule.

Methods well known to one skilled in the art may be used to administer the pharmaceutical composition of the present inevntion to patients, for example as intraarterial, intravenous, or percutaneous injections and also as intranasal, transbronchial, intramuscular or oral administrations. The dosage and method of administration vary according to the body-weight and age of a patient and the administration method; however, one skilled in the art can routinely select a suitable metod of administration. If said compound is encodable by a DNA, the DNA can be inserted into a vector for gene therapy and the vector administered to a patient to perform the therapy. The dosage and method of administration vary according to the body-weight, age, and symptoms of the patient but one skilled in the art can suitably select them.

For example, although the dose of a compound that binds to the protein of the present invention and regulates its activity depends on the symptoms, the dose is about 0.1 mg to about 100 mg per day, preferably about 1.0 mg to about 50 mg per day and more preferably about 1.0 mg to about 20 mg per day, when administered orally to a normal adult (weight 60 kg).

When administering parenterally, in the form of an injection to a normal adult (weight 60 kg), although there are some differences according to the patient, target organ, symptoms and method of administration, it is convenient to intravenously inject a dose of about 0.01 mg to about 30 mg per day, preferably about 0.1 to about 20 mg per day and more preferably about 0.1 to about 10 mg per day. Also, in the case of other animals too, it is possible to administer an amount converted to 60 kgs of body-weight.

Assessing the Prognosis of a Subject with DGC

Also provided is a method of assessing the prognosis of a subject with DGC by comparing the expression of one or more DGC-associated gene in a test cell population to the expression of the genes in a reference cell population derived from patients over a spectrum of disease stages. By comparing gene expression of one or more DGC-associated gene in the test cell population and the reference cell population(s), or by comparing the pattern of gene expression over time in test cell populations derived from the subject, the prognosis of the subject can be assessed.

A decrease in expression of one or more of DGC 137-463 compared to a normal control or an increase of expression of one or more of DGC 1-136 compared to a normal control indicates less favorable prognosis. A similar expression of one or more of DGC 1-463 indicates a more favorable prognosis compared to a normal control indicates a more favorable prognosis for the subject.

Kits

The invention also includes a DGC-detection reagent, e.g., a nucleic acid that specifically binds to or identifies one or more DGC nucleic acids such as oligonucleotide sequences, which are complementary to a portion of a DGC nucleic acid or antibodies which bind to proteins encoded by a DGC nucleic acid. The reagents are packaged together in the form of a kit. The reagents are packaged in separate containers, e.g., a nucleic acid or antibody (either bound to a solid matrix or packaged separately with reagents for binding them to the matrix), a control reagent (positive and/or negative), and/or a detectable label. Instructions (e.g., written, tape, VCR, CD-ROM, etc.) for carrying out the assay are included in the kit The assay format of the kit is a Northern hybridization or a sandwich ELISA known in the art.

For example, DGC detection reagent, is immobilized on a solid matrix such as a porous strip to form at least one DGC detection site. The measurement or detection region of the porous strip may include a plurality of sites containing a nucleic acid. A test strip may also contain sites for negative and/or positive controls. Alternatively, control sites are located on a separate strip from the test strip. Optionally, the different detection sites may contain different amounts of immobilized nucleic acids, i.e., a higher amount in the first detection site and lesser amounts in subsequent sites. Upon the addition of test sample, the number of sites displaying a detectable signal provides a quantitative indication of the amount of DGC present in the sample. The detection sites may be configured in any suitably detectable shape and are typically in the shape of a bar or dot spanning the width of a teststrip.

Alternatively, the kit contains a nucleic acid substrate array comprising one or more nucleic acids. The nucleic acids on the array specifically identify one or more nucleic acid sequences represented by DGC 1-463. The expression of 2, 3, 4, 5, 6, 7, 8, 9, 10, 15, 20, 25, 40 or 50 or more of the nucleic acids represented by DGC 1-463 are identified by virtue if the level of binding to an array test strip or chip. The substrate array can be on, e.g., a solid substrate, e.g., a “chip” as described in U.S. Pat. No. 5,744,305.

Arrays and Pluralities

The invention also includes a nucleic acid substrate array comprising one or more nucleic acids. The nucleic acids on the array specifically correspond to one or more nucleic acid sequences represented by DGC 1-463. The level expression of 2, 3, 4, 5, 6, 7, 8, 9, 10, 15, 20, 25, 40 or 50 or more of the nucleic acids represented by DGC 1-463 are identified by detecting nucleic acid binding to the array.

The invention also includes an isolated plurality (i.e., a mixture if two or more nucleic acids) of nucleic acid sequences. The nucleic acid sequence are in a liquid phase or a solid phase, e.g., immobilized on a solid support such as a nitrocellulose membrane. The plurality includes one or more of the nucleic acid sequences represented by DGC 1-463. In various embodiments, the plurality includes 2, 3, 4, 5, 6, 7, 8, 9, 10, 15, 20, 25, 40 or 50 or more of the sequences represented by DGC 1-463.

Chips

The DNA chip is a device that is convenient to compare expression levels of a number of genes at the same time. DNA chip-based expression profiling can be carried out, for example, by the method as disclosed in “Microarray Biochip Technology ” (Mark Schena, Eaton Publishing, 2000), etc.

A DNA chip comprises immobilized high-density probes to detect a number of genes. Thus, expression levels of many genes can be estimated at the same time by a single-round analysis. Namely, the expression profile of a specimen can be determined with a DNA chip. The DNA chip-based method of the present invention comprises the following steps of:

(1) synthesizing aRNAs or cDNAs corresponding to the marker genes;

(2) hybridizing the aRNAs or cDNAs with probes for marker genes; and

(3) detecting the aRNA or cDNA hybridizing with the probes and quantifying the amount of mRNA thereof.

The aRNA refers to RNA transcribed from a template cDNA with RNA polymerase. An aRNA transcription kit for DNA chip-based expression profiling is commercially available. With such a kit, aRNA can be synthesized from T7 promoter-attached cDNA as a template by using T7 RNA polymerase. On the other hand, by PCR using random primer, cDNA can be amplified using as a template a cDNA synthesized from mRNA.

On the other hand, the DNA chip comprises probes, which have been spotted thereon, to detect the marker genes of the present invention. There is no limitation on the number of marker genes spotted on the DNA chip. For example, it is allowed to select 5% or more, preferably 20% or more, more preferably 50% or more, still more preferably 70% or more of the marker genes of the present invention. Any other genes as well as the marker genes can be spotted on the DNA chip. For example, a probe for a gene whose expression level is hardly altered may be spotted on the DNA chip. Such a gene can be used to normalize assay results when assay results are intended to be compared between multiple chips or between different assays.

A probe is designed for each marker gene selected, and spotted on a DNA chip. Such a probe may be, for example, an oligonucleotide comprising 5-50 nucleotide residues. A method for synthesizing such oligonucleotides on a DNA chip is known to those skilled in the art. Longer DNAs can be synthesized by PCR or chemically. A method for spotting long DNA, which is synthesized by PCR or the like, onto a glass slide is also known to those skilled in the art. A DNA chip that is obtained by the method as described above can be used for diagnosing a DGC according to the present invention.

The prepared DNA chip is contacted with aRNA, followed by the detection of hybridization between the probe and aRNA. The aRNA can be previously labeled with a fluorescent dye. A fluorescent dye such as Cy3(red) and Cy5 (green) can be used to label an aRNA aRNAs from a subject and a control are labeled with different fluorescent dyes, respectively. The difference in the expression level between the two can be estimated based on a difference in the signal intensity. The signal of fluorescent dye on the DNA chip can be detected by a scanner and analyzed by using a special program. For example, the Suite from Affymetrix is a software package for DNA chip analysis.

Methods of Inhibiting DGC

The invention provides a method for treating a DGC in a subject. Therapeutic compounds are administered prophylactically or therapeutically to subject suffering from at risk of (or susceptible to) developing DGC. Such subjects are identified using standard clinical methods or by detecting an aberrant level of expression or activity of (e.g., DGC 1-463).

The therapeutic method includes increasing the expression, or function, or both of one or more gene products of genes whose expression is decreased (“under-expressed genes”) in a DGC cell relative to normal cells of the same tissue type from which the DGC cells are derived. In these methods, the subject is treated with an effective amount of a compound, which increases the amount of one of more of the under-expressed genes in the subject. Administration can be systemic or local. Therapeutic compounds include a polypeptide product of an under-expressed gene, or a biologically active fragment thereof a nucleic acid encoding an under-expressed gene and having expression control elements permitting expression in the DGC cells; for example an agent which increases the level of expression of such gene endogenous to the DGC cells (i.e., which up-regulates expression of the under-expressed gene or genes). Administration of such compounds counter the effects of aberrantly under-expressed of the gene or genes in the subjects gastric cells and improves the clinical condition of the subject.

The method also includes decreasing the expression, or function, or both, of one or more gene products of genes whose expression is aberrantly increased (“over-expressed gene”) in. Expression is inhibited in any of several ways known in the art For example, expression is inhibited by administering to the subject a nucleic acid that inhibits, or antagonizes, the expression of the over-expressed gene or genes, e.g., an antisense oligonucleotide or small interfering RNA which disrupts expression of the over-expressed gene or genes.

As noted above, antisense nucleic acids corresponding to the nucleotide sequence of DGC 1-136 can be used to reduce the expression level of the DGC 1-136. Antisense nucleic acids corresponding to DGC 1-136 that are up-regulated in DGC are useful for the treatment of DGC. Specifically, the antisense nucleic acids of the present invention may act by binding to the DGC 1-136 or mRNAs corresponding thereto, thereby inhibiting the transcription or translation of the genes, promoting the degradation of the mRNAs, and/or inhibiting the expression of proteins encoded by a nucleic acid selected from the group consisting of the DGC 1-136, finally inhibiting the function of the proteins. The term “antisense nucleic acids” as used herein encompasses both nucleotides that are entirely complementary to the target sequence and those having a mismatch of one or more nucleotides, so long as the antisense nucleic acids can specifically hybridize to the target sequences. For example, the antisense nucleic acids of the present invention include polynucleotides that have a homology of at least 70% or higher, preferably at 80% or higher, more preferably 90% or higher, even more preferably 95% or higher over a span of at least 15 continuous nucleotides. Algorithms known in the art can be used to determine the homology.

The antisense nucleic acid derivatives of the present invention act on cells producing the proteins encoded by marker genes by binding to the DNAs or mRNAs encoding the proteins, inhibiting their transcription or translation, promoting the degradation of the mRNAs, and inhibiting the expression of the proteins, thereby resulting in the inhibition of the protein function.

An antisense nucleic acid derivative of the present invention can be made into an external preparation, such as a liniment or a poultice, by mixing with a suitable base material which is inactive against the derivative.

Also, as needed, the derivatives can be formulated into tablets, powders, granules, capsules, liposome capsules, injections, solutions, nose-drops and freeze-drying agents by adding excipients, isotonic agents, solubilizers, stabilizers, preservatives, pain-killers, and such. These can be prepared by following known methods.

The antisense nucleic acids derivative is given to the patient by directly applying onto the ailing site or by injecting into a blood vessel so that it will reach the site of ailment. An antisense-mounting medium can also be used to increase durability and membrane-permeability. Examples are, liposomes, poly-L-lysine, lipids, cholesterol, lipofectin or derivatives of these.

The dosage of the antisense nucleic acid derivative of the present invention can be adjusted suitably according to the patient's condition and used in desired amounts. For example, a dose range of 0.1 to 100 mg/kg, preferably 0.1 to 50 mg/kg can be administered.

The antisense nucleic acids of the invention inhibit the expression of the protein of the invention and is thereby useful for suppressing the biological activity of a protein of the invention. Also, expression-inhibitors, comprising the antisense nucleic acids of the invention, are useful since they can inhibit the biological activity of a protein of the invention.

The antisense nucleic acids of present invention include modified oligonucleotides. For example, thioated nucleotides may be used to confer nuclease resistance to an oligonucleotide.

Also, a siRNA against marker gene can be used to reduce the expression level of the marker gene. By the term “siRNA” is meant a double stranded RNA molecule which prevents translation of a target mRNA. Standard techniques of introducing siRNA into the cell are used, including those in which DNA is a template from which RNA is transcribed. In the context of the present invention, the siRNA comprises a sense nucleic acid sequence and an anti-sense nucleic acid sequence against an up-regulated marker gene, such as DGC 1-136. The siRNA is constructed such that a single transcript has both the sense and complementary antisense sequences from the target gene, e.g., a hairpin.

The method is used to alter the expression in a cell of an up-regulated, e.g., as a result of malignant transformation of the cells. Binding of the siRNA to a transcript corresponding to one of the DGC 1-136 in the target cell results in a reduction in the protein production by the cell. The length of the oligonucleotide is at least 10 nucleotides and may be as long as the naturally-occurring the transcript. Preferably, the oligonucleotide is 19-25 nucleotides in length. Most preferably, the oligonucleotide is less than 75, 50, 25 nucleotides in length.

The nucleotide sequence of the siRNAs were designed using a siRNA design computer program available from the Ambion website (http://www.ambion.com/techlib/misc/siRNA_finder.html). The computer program selects nucleotide sequences for siRNA synthesis based on the following protocol.

Selection of siRNA Target Sites:

1. Beginning with the AUG start codon of the object transcript, scan downstream for AA dinucleotide sequences. Record the occurrence of each AA and the 3′ adjacent 19 nucleotides as potential siRNA target sites. Tuschl, et al. recommend against designing siRNA to the 5′ and 3′ untranslated regions (UTRs) and regions near the start codon (within 75 bases) as these may be richer in regulatory protein binding sites. UTR-binding proteins and/or translation initiation complexes may interfere with the binding of the siRNA endonuclease complex.

2. Compare the potential target sites to the human genome database and eliminate from consideration any target sequences with significant homology to other coding sequences. The homology search can be performed using BLAST, which can be found on the NCBI server at: www.ncbi.nlm.nih.gov/BLAST/

3. Select qualifying target sequences for synthesis. At Ambion, preferably several target sequences can be selected along the length of the gene for evaluation

The antisense oligonucleotide or siRNA of the invention inhibit the expression of the polypeptide of the invention and is thereby useful for suppressing the biological activity of the polypeptide of the invention. Also, expression-inhibitors, comprising the antisense oligonucleotide or siRNA of the invention, are useful in the point that they can inhibit the biological activity of the polypeptide of the invention. Therefore, a composition comprising the antisense oligonucleotide or siRNA of the present invention are useful in treating a DGC.

Alternatively, function of one or more gene products of the over-expressed genes is inhibited by administering a compound that binds to or otherwise inhibits the function of the gene products. For example, the compound is an antibody which binds to the over-expressed gene product, e.g., a cell surface protein or gene products and inhibits an activity of function of the gene product, e.g., binding to a cognate receptor.

The present invention refers to the use of antibodies, particularly antibodies against a protein encoded by an up-regulated marker gene, or a fragment of the antibody. As used herein, the term “antibody” refers to an immunoglobulin molecule having a specific structure, that interacts (i.e., binds) only with the antigen that was used for synthesizing the antibody (i.e., the up-regulated marker gene product) or with an antigen closely related to it. Furthermore, an antibody may be a fragment of an antibody or a modified antibody, so long as it binds to one or more of the proteins encoded by the marker genes. For instance, the antibody fragment may be Fab, F(ab′)₂, Fv, or single chain Fv (scFv), in which Fv fragments from H and L chains are ligated by an appropriate linker (Huston J. S. et al. Proc. Natl. Acad. Sci. U.S.A. 85:5879-5883 (1988)). More specifically, an antibody fragment may be generated by treating an antibody with an enzyme, such as papain or pepsin. Alternatively, a gene encoding the antibody fragment may be constructed, inserted into an expression vector, and expressed in an appropriate host cell (see, for example, Co M. S. et al. J. Immunol. 152:2968-2976 (1994); Better M. and Horwitz A. H. Methods Enzymol. 178:476-496 (1989); Pluckthun A. and Skerra A. Methods Enzymol. 178:497-515 (1989); Lamoyi E. Methods Enzymol. 121:652-663 (1986); Rousseaux J. et al. Methods Enzymol. 121:663-669 (1986); Bird R. E. and Walker B. W. Trends Biotechnol. 9:132-137 (1991)).

An antibody may be modified by conjugation with a variety of molecules, such as polyethylene glycol (PEG). The present invention provides such modified antibodies. The modified antibody can be obtained by chemically modifying an antibody. These modification methods are conventional in the field.

Alternatively, an antibody may be obtained as a chimeric antibody, between a variable region derived from a nonhuman antibody and a constant region derived from a human antibody, or as a humanized antibody, comprising the complementarity determining region (CDR) derived from a nonhuman antibody, the frame work region (FR) derived from a human antibody, and the constant region. Such antibodies can be prepared by using known technologies.

Cancer therapies directed at specific molecular alterations that occur in cancer cells have been validated through clinical development and regulatory approval of anti-cancer drugs such as trastuzumab (Herceptin) for the treatment of advanced breast cancer, imatinib methylate (Gleevec) for chronic myeloid leukemia, gefitinib (Iressa) for non-small cell lung cancer (NSCLC), and rituximab (anti-CD20 mAb) for B-cell lymphoma and mantle cell lymphoma (Ciardiello F, Tortora G. A novel approach in the treatment of cancer: targeting the epidermal growth factor receptor. Clin Cancer Res. October 2001;7(10):2958-70. Review.; Slamon D J, Leyland-Jones B, Shak S, Fuchs H, Paton V, Bajamonde A, Fleming T, Eiermann W, Wolter J, Pegram M, Baselga J, Norton L. Use of chemotherapy plus a monoclonal antibody against HER2 for metastatic breast cancer that overexpresses HER2. N Engl J Med. Mar. 15, 2001;344(11):783-92.; Rehwald U, Schulz H, Reiser M, Sieber M, Staak J O, Morschhauser F, Driessen C, Rudiger T, Muller-Hermelink K, Diehl V, Engert A. Treatment of relapsed CD20+ Hodgkin lymphoma with the monoclonal antibody rituximab is effective and well tolerated: results of a phase 2 trial of the German Hodgkin Lymphoma Study Group. Blood. Jan. 15, 2003;101(2):420-424.; Fang G, Kim C N, Perkins C L, Ramadevi N, Winton E, Wittmann S and Bhalla K N. (2000). Blood, 96, 2246-2253.). These drugs are clinically effective and better tolerated than traditional anti-cancer agents because they target only transformed cells. Hence, such drugs not only improve survival and quality of life for cancer patients, but also validate the concept of molecularly targeted cancer therapy. Furthermore, targeted drugs can enhance the efficacy of standard chemotherapy when used in combination with it (Gianni L. (2002). Oncology, 63 Suppl 1, 47-56.; Klejman A, Rushen L, Morrione A, Slupianek A and Skorski T. (2002). Oncogene, 21, 5868-5876.). Therefore, future cancer treatments will probably involve combining conventional drugs with target-specific agents aimed at different characteristics of tumor cells such as angiogenesis and invasiveness.

These modulatory methods are performed ex vivo or in vitro (e.g., by culturing the cell with the agent) or, alternatively, in vivo (e.g., by administering the agent to a subject). The method involves administering a protein or combination of proteins or a nucleic acid molecule or combination of nucleic acid, molecules as therapy to counteract aberrant expression or activity of the differentially expressed genes.

Diseases and disorders that are characterized by increased (relative to a subject not suffering from the disease or disorder) levels or biological activity of the genes may be treated with therapeutics that antagonize (i.e., reduce or inhibit) activity of the overexpressed gene or genes. Therapeutics that antagonize activity are administered therapeutically or prophylactically.

Therapeutics that may be utilized include, e.g., (i) a polypeptide, or analogs, derivatives, fragments or homologs thereof of the under-expressed sequence or sequences; (ii) antibodies to the over-expressed sequence or sequences; (iii) nucleic acids encoding the under-expressed sequence or sequences; (iv) antisense nucleic acids or nucleic acids that are “dysfunctional” (i.e., due to a heterologous insertion within the coding sequences of one or more over-expressed genes); or (v) small interfering RNA (siRNA); or (vi) modulators (i.e., inhibitors, agonists and antagonists that alter the interaction between an over/under-expressed polypeptide and its binding partner. The dysfunctional antisense molecules are utilized to “knockout” endogenous function of a polypeptide by homologous recombination (see, e.g., Capecchi, Science 244: 1288-1292 1989)

Diseases and disorders that are characterized by decreased (relative to a subject not suffering from the disease or disorder) levels or biological activity may be treated with therapeutics that increase (i.e., are agonists to) activity. Therapeutics that up-regulate activity may be administered in a therapeutic or prophylactic manner. Therapeutics that may be utilized include, but are not limited to, a polypeptide (or analogs, derivatives, fragments or homologs thereof) or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of a gene whose expression is altered). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, etc.).

Prophylactic administration occurs prior to the manifestation of overt clinical symptoms of disease, such that a disease or disorder is prevented or, alternatively, delayed in its progression.

Therapeutic methods include contacting a cell with an agent that modulates one or more of the activities of the gene products of the differentially expressed genes. An agent that modulates protein activity includes a nucleic acid or a protein, a naturally-occurring cognate ligand of these proteins, a peptide, a peptidomimetic, or other small molecule. For example, the agent stimulates one or more protein activities of one or more of a differentially under-expressed gene.

The present invention also relates to a method of treating or preventing DGC in a subject comprising administering to said subject a vaccine comprising a polypeptide encoded by a nucleic acid selected from the group consisting of DGC 1-136 or an immunologically active fragment of said polypeptide, or a polynucleotide encoding the polypeptide or the fragment thereof. An administration of the polypeptide induce an anti-tumor immunity in a subject. To inducing anti-tumor immunity, a polypeptide encoded by a nucleic acid selected from the group consisting of DGC 1-136 or an immunologically active fragment of said polypeptide, or a polynucleotide encoding the polypeptide is administered. The polypeptide or the immunologically active fragments thereof are useful as vaccines against DGC. In some cases the proteins or fragments thereof may be administered in a form bound to the T cell recepor (TCR) or presented by an antigen presenting cell (APC), such as macrophage, dendritic cell (DC), or B-cells. Due to the strong antigen presenting ability of DC, the use of DC is most preferable among the APCs.

In the present invention, vaccine against DGC refers to a substance that has the function to induce anti-tumor immunity upon inoculation into animals. According to the present invention, polypeptides encoded by a nucleic acid selected from the group consisting of DGC 1-136 or fragments thereof were suggested to be HLA-A24 or HLA-A*0201 restricted epitopes peptides that may induce potent and specific immune response against DGC cells expressing DGC 1-136. Thus, the present invention also encompasses method of inducing anti-tumor immunity using the polypeptides. In general, anti-tumor immunity includes immune responses such as follows:

induction of cytotoxic lymphocytes against tumors,

induction of antibodies that recognize tumors, and

induction of anti-tumor cytokine production.

Therefore, when a certain protein induces any one of these immune responses upon inoculation into an animal, the protein is decided to have anti-tumor immunity inducing effect. The induction of the anti-tumor immunity by a protein can be detected by observing in vivo or in vitro the response of the immune system in the host against the protein.

For example, a method for detecting the induction of cytotoxic T lymphocytes is well known. A foreign substance that enters the living body is presented to T cells and B cells by the action of antigen presenting cells (APCs). T cells that respond to the antigen presented by APC in antigen specific manner differentiate into cytotoxic T cells (or cytotoxic T lymphocytes; CTLs) due to stimulation by the antigen, and then proliferate (this is referred to as activation of T cells). Therefore, CTL induction by a certain peptide can be evaluated by presenting the peptide to T cell by APC, and detecting the induction of CTL. Furthermore, APC has the effect of activating CD4+ T cells, CD8+ T cells, macrophages, eosinophils, and NK cells. Since CD4+ T cells and CD8+ T cells are also important in anti-tumor immunity, the anti-tumor immunity inducing action of the peptide can be evaluated using the activation effect of these cells as indicators.

A method for evaluating the inducing action of CTL using dendritic cells (DCs) as APC is well known in the art. DC is a representative APC having the strongest CTL inducing action among APCs. In this method, the test polypeptide is initially contacted with DC, and then this DC is contacted with T cells. Detection of T cells having cytotoxic effects against the cells of interest after the contact with DC shows that the test polypeptide has an activity of inducing the cytotoxic T cells. Activity of CTL against tumors can be detected, for example, using the lysis of ⁵¹Cr-labeled tumor cells as the indicator. Alternatively, the method of evaluating the degree of tumor cell damage using ³H-thymidine uptake activity or LDH (lactose dehydrogenase)-release as the indicator is also well known.

Apart from DC, peripheral blood mononuclear cells (PBMCs) may also be used as the APC. The induction of CTL is reported that the it can be enhanced by culturing PBMC in the presence of GM-CSF and IL-4. Similarly, CTL has been shown to be induced by culturing PBMC in the presence of keyhole limpet hemocyanin (KLH) and IL-7.

The test polypeptides confirmed to possess CTL inducing activity by these methods are polypeptides having DC activation effect and subsequent CTL inducing activity. Therefore, polypeptides that induce CTL against tumor cells are useful as vaccines against tumors. Furthermore, APC that acquired the ability to induce CTL against tumors by contacting with the polypeptides are useful as vaccines against tumors. Furthermore, CTL that acquired cytotoxicity due to presentation of the polypeptide antigens by APC can be also used as vaccines against tumors. Such therapeutic methods for tumors using anti-tumor immunity due to APC and CTL are referred to as cellular immunotherapy.

Generally, when using a polypeptide for cellular immunotherapy, efficiency of the CTL-induction is known to increase by combining a plurality of polypeptides having different structures and contacting them with DC. Therefore, when stimulating DC with protein fragments, it is advantageous to use a mixture of multiple types of fragments.

Alternatively, the induction of anti-tumor immunity by a polypeptide can be confirmed by observing the induction of antibody production against tumors. For example, when antibodies against a polypeptide are induced in a laboratory animal immunized with the polypeptide, and when growth of tumor cells is suppressed by those antibodies, the polypeptide can be determined to have an ability to induce anti-tumor immunity.

Anti-tumor immunity is induced by administering the vaccine of this invention, and the induction of anti-tumor immunity enables treatment and prevention of DGC. Therapy against cancer or prevention of the onset of cancer includes any of the steps, such as inhibition of the growth of cancerous cells, involution of cancer, and suppression of occurrence of cancer. Decrease in mortality of individuals having cancer, decrease of tumor markers in the blood, alleviation of detectable symptoms accompanying cancer, and such are also included in the therapy or prevention of cancer. Such therapeutic and preventive effects are preferably statistically significant For example, in observation, at a significance level of 5% or less, wherein the therapeutic or preventive effect of a vaccine against cell proliferative diseases is compared to a control without vaccine administration. For example, Student's t-test, the Mann-Whitney U-test, or ANOVA may be used for statistical analyses.

The above-mentioned protein having immunological activity or a vector encoding the protein may be combined with an adjuvant. An adjuvant refers to a compound that enhances the immune response against the protein when administered together (or successively) with the protein having immunological activity. Examples of adjuvants include cholera toxin, salmonella toxin, alum, and such, but are not limited thereto. Furthermore, the vaccine of this invention may be combined appropriately with a pharmaceutically acceptable carrier. Examples of such carriers are sterilized water, physiological saline, phosphate buffer, culture fluid, and such. Furthermore, the vaccine may contain as necessary, stabilizers, suspensions, preservatives, surfactants, and such. The vaccine is administered systemically or locally. Vaccine administration may be performed by single administration, or boosted by multiple administrations.

When using APC or CTL as the vaccine of this invention, tumors can be treated or prevented, for example, by the ex vivo method. More specifically, PBMCs of the subject receiving treatment or prevention are collected, the cells are contacted with the polypeptide ex vivo, and following the induction of APC or CTL, the cells may be administered to the subject. APC can be also induced by introducing a vector encoding the polypeptide into PBMCs ex vivo. APC or CTL induced in vitro can be cloned prior to administration. By cloning and growing cells having high activity of damaging target cells, cellular immunotherapy can be performed more effectively. Furthermore, APC and CTL isolated in this manner may be used for cellular immunotherapy not only against individuals from whom the cells are derived, but also against similar types of tumors from other individuals.

Furthermore, a pharmaceutical composition for treating or preventing a cell proliferative disease, such as cancer, comprising a pharmaceutically effective amount of the polypeptide of the present invention is provided. The pharmaceutical composition may be used for raising anti tumor immunity.

Pharmaceutical Compositions for Inhibiting DGC

Pharmaceutical formulations include those suitable for oral, rectal, nasal, topical (including buccal and sub-lingual), vaginal or parenteral (including intramuscular, sub-cutaneous and intravenous) administration, or for administration by inhalation or insufflation. The formulations are optionally packaged in discrete dosage units

Pharmaceutical formulations suitable for oral administration include capsules, cachets or tablets, each containing a predetermined amount of the active ingredient. Formulations also include powders, granules or solutions, suspensions or emulsions. The active ingredient is optionally administered as a bolus electuary or paste. Tablets and capsules for oral administration may contain conventional excipients such as binding agents, fillers, lubricants, disintegrant or wetting agents. A tablet may be made by compression or molding, optionally with one or more formulational ingredients. Compressed tablets may be prepared by compressing in a suitable machine the active ingredients in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may be coated according to methods well known in the art. Oral fluid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives. The tablets may optionally be formulated so as to provide slow or controlled release of the active ingredient therein. A package of tablets may contain one tablet to be taken on each of the month. The formulation or does of medicament varies with respect to the phase (probe or sucretary) of the menstrual cycle.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit dose or multi-dose containers, for example sealed ampoules and vials, and may be stored in a freeze-dried (lyophilized) condition requiring only the addition of the sterile liquid carrier, for example, saline, water-for-injection, immediately prior to use. Alternatively, the formulations may be presented for continuous infusion. Extemporaneous injection solutions and suspensions may be prepared from sterile powders, granules and tablets of the kind previously described.

Formulations for rectal administration include suppositories with standard carriers such as cocoa butter or polyethylene glycol. Formulations for topical administration in the mouth, for example buccally or sublingually, include lozenges, which contain the active ingredient in a flavored base such as sucrose and acacia or tragacanth, and pastilles comprising the active ingredient in a base such as gelatin and glycerin or sucrose and acacia For intra-nasal administration the compounds of the invention may be used as a liquid spray or dispersible powder or in the form of drops. Drops may be formulated with an aqueous or non-aqueous base also comprising one or more dispersing agents, solubilizing agents or suspending agents.

For administration by inhalation the compounds are conveniently delivered from an insufflator, nebulizer, pressurized packs or other convenient means of delivering an aerosol spray. Pressurized packs may comprise a suitable propellant such as dichlorodifluoromethane, trichlorofluoromethane, dichiorotetrafluoroethane, carbon dioxide or other suitable gas. In the case of a pressurized aerosol, the dosage unit may be determined by providing a valve to deliver a metered amount.

Alternatively, for administration by inhalation or insufflation, the compounds may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflators.

Other formulations include implantable devices and adhesive patches; which release a therapeutic agent.

When desired, the above described formulations, adapted to give sustained release of the active ingredient, may be employed. The pharmaceutical compositions may also contain other active ingredients such as antimicrobial agents, immunosuppressants or preservatives.

It should be understood that in addition to the ingredients particularly mentioned above, the formulations of this invention may include other agents conventional in the art having regard to the type of formulation in question, for example, those suitable for oral administration may include flavoring agents.

Preferred unit dosage formulations are those containing an effective dose, as recited below, or an appropriate fraction thereof, of the active ingredient.

For each of the aforementioned conditions, the compositions, e.g., polypeptides and organic compounds are administered orally or via injection at a dose of from about 0.1 to about 250 mg/kg per day. The dose range for adult humans is generally from about 5 mg to about 17.5 g/day, preferably about 5 mg to about 10 g/day, and most preferably about 100 mg to about 3 g/day. Tablets or other unit dosage forms of presentation provided in discrete units may conveniently contain an amount which is effective at such dosage or as a multiple of the same, for instance, units containing about 5 mg to about 500 mg, usually from about 100 mg to about 500 mg. Nucleic acids, e.g., DNA constructs, are administered at a dose in the range of 0.005-50 mg/kg of body weight. Alternatively, an intervenous dose is in the range of 106-1022 copies if the nucleic acid molecule.

The dose employed will depend upon a number of factors, including the age and sex of the subject, the precise disorder being treated, and its severity. Also the route of administration may vary depending upon the condition and its severity.

The invention will be further described in the following examples, which do not limit the scope of the invention described in the claims. The following examples illustrate the identification and characterization of genes differentially expressed in DGC cells.

### EXAMPLE 1

### Patients and Tissue Samples

Tissue obtained from diseased tissue (e.g., mucosae from DGC) and normal tissues was evaluated to identify genes which are differently expressed or a disease state, e.g., DGC. The assays were carried out as follows.

Primary gastric cancers and corresponding non-cancerous gastric mucosae were obtained with informed consent from 20 patients who underwent gastrectomy. Patient profiles were obtained from medical records. Histopathological classification of each tumor, performed according to the Lauren's classification (2), diagnosed all samples as diffuse-type gastric adenocarcinomas. Clinical stage was determined according to the UICC TNM classification. The 20 gastric cancer tissues included 19 advanced (T2-T4) cancers and one early (T1) cancer. All samples were immediately frozen and embedded in TissueTek OCT medium (Sakura, Tokyo, Japan) and stored at −80° C. until used for microarray analysis.

Laser-Microbeam Microdissection, Extraction of RNA, and T7-Based RNA Amplification

The frozen sections were prepared, fixed in 70% ethanol for 45 sec., stained with hematoxylin and eosin, and dehydrated in 70%, 80%, and 90% ethanol for 30 sec. of each step, followed by a final dehydration in 100% ethanol for two min. Once air-dried, cancer cells and non-cancerous gastric epithelium were selectively collected from the stained tissues using laser-microbeam microdissection. Extraction of total RNA and T7-based amplification were performed using methods known in the art (8). 2.5-μg aliquots of twice-amplified RNA (aRNA) from each cancerous and non-cancerous tissue were labeled with Cy3-dCTP and Cy5-dCTP respectively.

cDNA Microarray and Analysis of Data

Fabrication of the cDNA microarray slides, hybridization, washing and detection of signals were carried out using methods known in the art (8). The fluorescence intensities of Cy5 (non-tumor) and Cy3 (tumor) for each target spot were adjusted so that the mean Cy3/Cy5 ratios of 52 housekeeping genes were equal to one. Cut-off values for signal intensities were determined on each slide so that all filtered genes have greater S/N (signal to noise) ratios of Cy3 or Cy5 than three, and then excluded genes for further analysis when both Cy3 and Cy5 dyes gave signal intensities lower than the cut-off values. Genes were categorized into three groups according to their expression ratios (Cy3/Cy5): up-regulated (ratio equal to or greater than 2.0), down-regulated (ratio equal to or less than 0.3), and unchanged expression (ratios between 0.3 and 2.0). Genes with Cy3/Cy5 ratios greater than 2.0 or less than 0.3 in more than 50% of the cases examined were defined as commonly up- or down-regulated genes, respectively.

Semi-Quantitative RT-PCR

Five commonly up-regulated genes (TGFBI, SPARC, COL3A1, MSLN, and an EST were selected and their expression levels were examined by semi-quantitative RT-PCR. The FDFT1 gene served as an internal control because it showed the smallest Cy3/Cy5 fluctuation among the 52 housekeeping genes in our experiments. The PCR reaction was preceded by 95° C. for 2 min, then underwent 25 cycles of 95° C. for 30 s, 60° C. for 30 s, and 72° C. for 30 s followed by final extension of 72° C. for 5 min. The sequences of primers were as follows:

FDET1
forward primer,

(SEQ ID NO:1)

5′-TGTGTGGCTGGGACCTTTAGGAA-3′,
and
reverse,

(SEQ ID NO:2)

3′-TCATTCTAGCCAGGATCATACTAAG-5′;
TGFBI
forward primer,

(SEQ ID NO:3)

5′-TCCCTGGAAAAGGAGCTTCAGTA-3′,
and
reverse,

(SEQ ID NO:4)

3′-ACACCATGGCTCTGTCACAATAG-5′;
SPARC
forward primer,

(SEQ ID NO:5)

5′-CAAGAGTGAGATGTAGAAAGTTGT-3′
and
reverse,

(SEQ ID NO:6)

3′-CTTCACATCATGGTGAGAGTTTG-5′;
COL3A1
forward primer,

(SEQ ID NO:7)

5′-AGACGCATGTTATGGTGCTAATGTA-3′
and
reverse,

(SEQ ID NO:8)

3′-GATCAACAACCACATACAAGCTTAC-5′;
MSLN
forward primer,

(SEQ ID NO:9)

5′-AACGGCTACCTGGTCCTAGAC-3′
and
reverse,

(SEQ ID NO:10)

3′-GTTTACTGAGCGCGAGTTCTCT-5′;
an EST (Genbank Accession No.AA430699)
forward primer,

(SEQ ID NO:11)

5′-TTTAACGCTGGTGGGCAGCA-3′
and
reverse,

(SEQ ID NO:12)

3′-ATAAACAGAACCCATCCCAAG-5′.

### EXAMPLE 2

### Identification of Genes with Clinically Relevant Expression Patterns in DGC Cells

To clarify mechanisms underlying carcinogenesis of the DGC, genes that were commonly up- or down-regulated in this type of tumor were searched. A cDNA microarray analysis of more than 20,000 genes in 20 tumors identified 136 genes that were up-regulated in more than 50% of the cases examined (Table 1). 327 genes that were down-regulated in 50% or more of the samples examined were also identified (Table 2).

Commonly up-regulated elements included genes associated with signal-transduction pathways (TGFBI, ARHGDIB, and GNAI2), genes encoding transcription factors (HMG1Y), and genes involved in various metabolic pathways (AHCY, IMPDH2, and GNPI), transport systems (SLC20A1), apotosis (NOD1), protein translation and processing (EIF3S6, CCT2, HSPCB, and HSPB1), DNA replication and recombination (CDC25B).

Among the commonly down-regulated genes were some that are involved in carbohydrate metabolism (ADH3, ALDH3, FBP1, and ADH1), drug metabolism (CYP3A7, and CYP3A5), carbon dioxide metabolism (CA2), defense response (TFF1, TFF2) or transport of small molecules or heavy metals (ATP4A, ATP4B, ATP2A3, GIF, MT1E, MT1L).

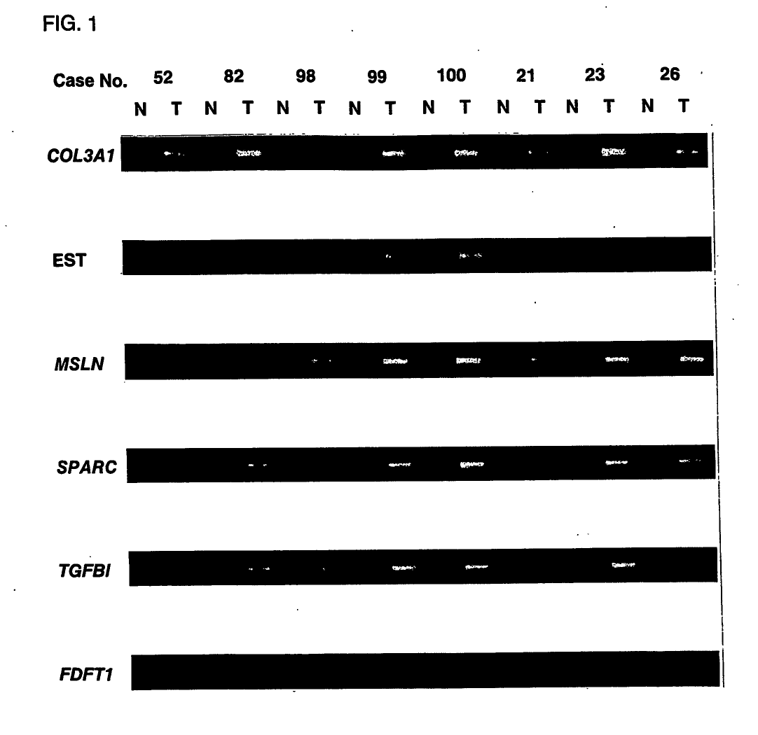

To verify the microarray data, five commonly up-regulated genes (TGFBI, SPARC, COL3A1, MSLN, and an EST (Genbank Accession No.AA430699) were selected and semi-quantitative RT-PCR using eight pairs of RNAs used for microarray was performed. The results confirmed microarray data for all five genes (FIG. 1), supporting the reliability of the diagnostic assay using the DGC genes described herein.

TABLE 1
Up-regulated genes in diffuse-type gastric cancer

| DGC | | | |
|---|---|---|---|
| Assignment | ACCESSION | GENE | TITLE |
| 1 | D16294 | ACAA2 | acetyl-Coenzyme A acyltransferase 2 |
| | | | (mitochondrial 3-oxoacyl-Coenzyme A thiolase) |
| 2 | M18112 | ADPRT | ADP-ribosyltransferase (NAD+; poly (ADP- |
| | | | ribose) polymerase) |
| 3 | M61831 | AHCY | S-adenosylhomocysteine hydrolase |
| 4 | X05908 | ANXA1 | annexin A1 |
| 5 | D00017 | ANXA2 | annexin A2 |
| 6 | D00172 | ANXA5 | annexin A5 |
| 7 | U25182 | AOE372 | thioredoxin peroxidase (antioxidant enzyme) |
| 8 | L20688 | ARHGDIB | Rho GDP dissociation inhibitor (GDI) beta |
| 9 | U51478 | ATP1B3 | ATPase, Na+/K+ transporting, beta 3 |
| | | | polypeptide |
| 10 | U75285 | BIRC5 | baculoviral IAP repeat-containing 5 (survivin) |
| 11 | AA634515 | CCT2 | chaperonin containing TCP1, subunit 2 (beta) |
| 12 | M94083 | CCT6A | chaperonin containing TCP1, subunit 6A (zeta 1) |
| 13 | K01144 | CD74 | CD74 antigen (invariant polypeptide of major |
| | | | histocompatibility complex, class II antigen- |
| | | | associated) |
| 14 | M33680 | CD81 | CD81 antigen (target of antiproliferative |
| | | | antibody 1) |
| 15 | AA421724 | CDC20 | CDC20 (cell division cycle 20, S. cerevisiae, |
| | | | homolog) |
| 16 | M81934 | CDC25B | cell division cycle 25B |
| 17 | AA621571 | CLDN7 | claudin 7 |
| 18 | X91788 | CLNS1A | chloride channel, nucleotide-sensitive, 1A |
| 19 | AA977821 | COL1A1 | collagen, type I, alpha 1 |
| 20 | J03464 | COL1A2 | collagen, type I, alpha 2 |
| 21 | X14420 | COL3A1 | collagen, type III, alpha 1 (Ehlers-Danlos |
| | | | syndrome type IV, autosomal dominant) |
| 22 | X03963 | COL4A1 | collagen, type IV, alpha 1 |
| 23 | X05610 | COL4A2 | collagen, type IV, alpha 2 |
| 24 | M30448 | CSNK2B | casein kinase 2, beta polypeptide |
| 25 | AA985222 | CTSB | cathepsin B |
| 26 | AA143048 | DKFZP564O0463 | DKFZP564O0463 protein |
| 27 | AW276358 | DPYSL2 | dihydropyrimidinase-like 2 |
| 28 | U41515 | DSS1 | Deleted in split-hand/split-foot 1 region |
| 29 | R61297 | EIF3S6 | eukaryotic translation initiation factor 3, subunit |
| | | | 6 (48 kD) |
| 30 | AF010314 | ENC1 | ectodermal-neural cortex (with BTB-like |
| | | | domain) |
| 31 | M14328 | ENO1 | enolase 1, (alpha) |
| 32 | BE439695 | EPB72 | erythrocyte membrane protein band 7.2 |
| | | | (stomatin) |
| 33 | D12765 | ETV4 | ets variant gene 4 (E1A enhancer-binding |
| | | | protein, E1AF) |
| 34 | U07424 | FARSL | phenylalanine-tRNA synthetase-like |
| 35 | AI139231 | FBL | fibrillarin |
| 36 | AA761293 | FKBP1A | FK506 binding protein 1A (12 kD) |
| 37 | AI394016 | FLJ20116 | hypothetical protein FLJ20116 |
| 38 | AA703211 | FLJ20736 | hypothetical protein FLJ20736 |
| 39 | X02761 | FN1 | fibronectin 1 |
| 40 | M33197 | GAPD | glyceraldehyde-3-phosphate dehydrogenase |
| 41 | J03004 | GNAI2 | guanine nucleotide binding protein (G protein), |
| | | | alpha inhibiting activity polypeptide 2 |
| 42 | D31766 | GNPI | glucosamine-6-phosphate isomerase |
| 43 | M21304 | GPX1 | glutathione peroxidase 1 |
| 44 | X71973 | GPX4 | glutathione peroxidase 4 (phospholipid |
| | | | hydroperoxidase) |
| 45 | U21242 | GTF2A2 | general transcription factor IIA, 2 (12 kD subunit) |
| 46 | M76766 | GTF2B | general transcription factor IIB |
| 47 | AA652197 | GW112 | differentially expressed in hematopoietic |
| | | | lineages |
| 48 | U91316 | HBACH | cytosolic acyl coenzyme A thioester hydrolase |
| 49 | AA583491 | HCA112 | hepatocellular carcinoma-associated antigen 112 |
| 50 | AA043590 | HECH | heterochromatin-like protein 1 |
| 51 | AA714394 | HMG2 | high-mobility group (nonhistone chromosomal) |
| | | | protein 2 |
| 52 | L17131 | HMG1Y | high-mobility group (nonhistone chromosomal) |
| | | | protein isoforms I and Y |
| 53 | D66904 | HRMT1L2 | HMT1 (hnRNP methyltransferase, S. cerevisiae)- |
| | | | like 2 |
| 54 | AW084318 | HSPB1 | heat shock 27 kD protein 1 |
| 55 | AI268685 | HSPC023 | HSPC023 protein |
| 56 | AI273886 | HSPCB | heat shock 90 kD protein 1, beta |
| 57 | AI081175 | IFITM1 | interferon induced transmembrane protein 1 (9-27) |
| 58 | X57351 | IFITM2 | interferon induced transmembrane protein 2 (1-8D) |
| 59 | M87789 | IGHG3 | immunoglobulin heavy constant gamma 3 (G3m |
| | | | marker) |
| 60 | J04208 | IMPDH2 | IMP (inosine monophosphate) dehydrogenase 2 |
| 61 | M13755 | ISG15 | interferon-stimulated protein, 15 kDa |
| 62 | AB003184 | ISLR | immunoglobulin superfamily containing leucine- |
| | | | rich repeat |
| 63 | X07979 | ITGB1 | integrin, beta 1 (fibronectin receptor, beta |
| | | | polypeptide, antigen CD29 includes MDF2, |
| | | | MSK12) |
| 64 | M85217 | KCNA3 | potassium voltage-gated channel, shaker-related |
| | | | subfamily, member 3 |
| 65 | D43950 | KIAA0098 | Homo sapiens PNAS02 mRNA, complete cds |
| 66 | D21853 | KIAA0111 | KIAA0111 gene product |
| 67 | AA394063 | KIAA0144 | KIAA0144 gene product |
| 68 | D63486 | KIAA0152 | KIAA0152 gene product |
| 69 | AA811263 | KIAA1268 | KIAA1268 protein |
| 70 | X03212 | KRT7 | keratin 7 |
| 71 | X53305 | LAP18 | leukemia-associated phosphoprotein p18 |
| | | | (stathmin) |
| 72 | AA742701 | LCP1 | lymphocyte cytosolic protein 1 (L-plastin) |
| 73 | X03445 | LMNA | lamin A/C |
| 74 | W74416 | LOC51126 | N-terminal acetyltransferase complex |
| | | | ard1subunit |
| 75 | AA477299 | LOC51202 | hqp0256 protein |
| 76 | U42376 | LY6E | lymphocyte antigen 6 complex, locus E |
| 77 | AC005546 | LYL1 | lymphoblastic leukemia derived sequence 1 |
| 78 | L10612 | MIF | macrophage migration inhibitory factor |
| | | | (glycosylation-inhibiting factor) |
| 79 | AU155489 | MMP7 | matrix metalloproteinase 7 (matrilysin, uterine) |
| 80 | D49441 | MSLN | mesothelin |
| 81 | U46920 | MTX1 | metaxin 1 |
| 82 | X17620 | NME1 | non-metastatic cells 1, protein (NM23A) |
| | | | expressed in |
| 83 | U20971 | NNMT | nicotinamide N-methyltransferase |
| 84 | AA242961 | NOD1 | caspase recruitment domain 4 |
| 85 | Y09022 | NOT56L | Not56 (D. melanogaster)-like protein |
| 86 | U02020 | PBEF | pre-B-cell colony-enhancing factor |
| 87 | AI265770 | PDLIM1 | PDZ and LIM domain 1 (elfin) |
| 88 | S85655 | PHB | prohibitin |
| 89 | N30179 | PLAB | prostate differentiation factor |
| 90 | AF001601 | PON2 | paraoxonase 2 |
| 91 | AA625878 | PPIA | peptidylprolyl isomerase A (cyclophilin A) |
| 92 | U44772 | PPT1 | palmitoyl-protein thioesterase 1 (ceroid- |
| | | | lipofuscinosis, neuronal 1, infantile) |
| 93 | AF044588 | PRC1 | protein regulator of cytokinesis 1 |
| 94 | AA670141 | PRKDC | protein kinase, DNA-activated, catalytic |
| | | | polypeptide |
| 95 | AA346311 | RAI3 | retinoic acid induced 3 |
| 96 | D42073 | RCN1 | reticulocalbin 1, EF-hand calcium binding |
| | | | domain |
| 97 | L11566 | RPL18 | ribosomal protein L18 |
| 98 | U14969 | RPL28 | ribosomal protein L28 |
| 99 | AA316619 | RPL30 | ribosomal protein L30 |
| 100 | J02984 | RPS15 | ribosomal protein S15 |
| 101 | AA308139 | S100A10 | S100 calcium-binding protein A10 (annexin II |
| | | | ligand, calpactin I, light polypeptide (p11)) |
| 102 | AF039690 | SDCCAG8 | serologically defined colon cancer antigen 8 |
| 103 | K02215 | SERPINA8 | serine (or cysteine) proteinase inhibitor, clade A |
| | | | (alpha antiproteinase, antitrypsin), member 8 |
| 104 | M13690 | SERPING1 | serine (or cysteine) proteinase inhibitor, clade G |
| | | | (C1 inhibitor), member 1 |
| 105 | L11932 | SHMT1 | serine hydroxymethyltransferase 1 |
| 106 | L20859 | SLC20A1 | solute carrier family 20 (phosphate transporter), |
| | | | member 1 |
| 107 | J03040 | SPARC | secreted protein, acidic, cysteine-rich |
| | | | (osteonectin) |
| 108 | L15203 | TFF3 | trefoil factor 3 (intestinal) |
| 109 | M77349 | TGFBI | transforming growth factor, beta-induced, 68 kD |
| 110 | AI049960 | TGIF2 | TGFB-induced factor 2 (TALE family |
| | | | homeobox) |
| 111 | M12670 | TIMP1 | tissue inhibitor of metalloproteinase 1 (erythroid |
| | | | potentiating activity, collagenase inhibitor) |
| 112 | AA536113 | TMEPAI | transmembrane, prostate androgen induced RNA |
| 113 | AF004430 | TPD52L2 | tumor protein D52-like 2 |
| 114 | M33492 | TPSB1 | tryptase beta 1 |
| 115 | U45328 | UBE2I | ubiquitin-conjugating enzyme E2I (homologous |
| | | | to yeast UBC9) |
| 116 | U44839 | USP11 | ubiquitin specific protease 11 |
| 117 | X94991 | ZYX | zyxin |
| 118 | AA449335 | | ESTs |
| 119 | W93907 | | Putative integral membrane transporter |
| 120 | T74135 | | ESTs |
| 121 | AA430699 | | ESTs |
| 122 | N49596 | | Homo sapiens cDNA FLJ12179 fis, clone |
| | | | MAMMA1000738, moderately similar to |
| | | | HYPOTHETICAL 116.5 KD PROTEIN |
| | | | C20G8.09C IN CHROMOSOME I |
| 123 | AA143060 | | ESTs, Highly similar to I38945 melanoma |
| | | | ubiquitous mutated protein [H. sapiens] |
| 124 | AA369905 | | ESTs |
| 125 | AA455877 | | Homo sapiens cDNA FLJ11177 fis, clone |
| | | | PLACE1007402 |
| 126 | AI755112 | | Human betaD integrin mRNA, cytoplasmic |
| | | | domain, partial cds |
| 127 | W94363 | | ESTs, Weakly similar to ALU4_HUMAN ALU |
| | | | SUBFAMILY SB2 SEQUENCE |
| | | | CONTAMINATION WARNING ENTRY |
| | | | [H. sapiens] |
| 128 | N95414 | | ESTs |
| 129 | AA633908 | | ESTs |
| 130 | AA885480 | | Human DNA sequence from clone RP5-858B6 |
| | | | on chromosome 1q42.13-43 Contains ESTs, |
| | | | STSs, GSSs and a CpG island. Contains three |
| | | | novel genes |
| 131 | N36716 | | ESTs |
| 132 | AA416843 | | ESTs |
| 133 | T55019 | | ESTs, fetal spleen |
| 134 | AA412367 | | ESTs, Weakly similar to ORF YGL050w |
| | | | [S. cerevisiae] |
| 135 | AA149846 | | Homo sapiens mRNA; cDNA DKFZp762B195 |
| | | | (from clone DKFZp762B195) |
| 136 | AI300800 | | ESTs, Weakly similar to RL22_HUMAN 60S |
| | | | RIBOSOMAL PROTEIN L22 [H. sapiens] |

TABLE2
Down-regulated genes in diffuse-type gastric cancer

| DGC | | | |
|---|---|---|---|
| Assignment | ACCESSION | GENE | TITLE |
| 137 | U57961 | 13CDNA73 | putative gene product |
| 138 | AI022193 | A1BG | alpha-B glycoprotein |
| 139 | L05628 | ABCC1 | ATP-binding cassette, sub-family C |
| | | | (CFTR/MRP), member 1 |
| 140 | S69189 | ACOX1 | acyl-Coenzyme A oxidase 1, palmitoyl |
| 141 | J00068 | ACTA1 | actin, alpha 1, skeletal muscle |
| 142 | M12963 | ADH1 | alcohol dehydrogenase 1 (class I), alpha |
| | | | polypeptide |
| 143 | X04299 | ADH3 | alcohol dehydrogenase 3 (class I), gamma |
| | | | polypeptide |
| 144 | D29952 | ADRA1D | adrenergic, alphaD-, receptor |
| 145 | AF044961 | AKR1B11 | aldo-keto reductase family 1, member B11 |
| | | | (aldose reductase-like) |
| 146 | U05861 | AKR1C1 | aldo-keto reductase family 1, member C1 |
| | | | (dihydrodiol dehydrogenase 1; 20-alpha (3- |
| | | | alpha)-hydroxysteroid dehydrogenase) |
| 147 | D17793 | AKR1C3 | aldo-keto reductase family 1, member C3 (3- |
| | | | alpha hydroxysteroid dehydrogenase, type II) |
| 148 | D26125 | AKR1C4 | aldo-keto reductase family 1, member C4 |
| | | | (chlordecone reductase; 3-alpha hydroxysteroid |
| | | | dehydrogenase, type I; dihydrodiol |
| | | | dehydrogenase 4) |
| 149 | AF026947 | AKR7A2 | aldo-keto reductase family 7, member A2 |
| | | | (aflatoxin aldehyde reductase) |
| 150 | M77477 | ALDH3 | aldehyde dehydrogenase 3 |
| 151 | M22324 | ANPEP | alanyl (membrane) aminopeptidase |
| | | | (aminopeptidase N, aminopeptidase M, |
| | | | microsomal aminopeptidase, CD13, p150) |
| 152 | T92046 | APBB2 | amyloid beta (A4) precursor protein-binding, |
| | | | family B, member 2 (Fe65-like) |
| 153 | U48408 | AQP6 | aquaporin 6, kidney specific |
| 154 | AF049884 | ARGBP2 | Arg/Abl-interacting protein ArgBP2 |
| 155 | AA677562 | ARHF | Ras homolog gene family, member F (in |
| | | | filopodia) |
| 156 | M15798 | ASNS | asparagine synthetase |
| 157 | Y15724 | ATP2A3 | ATPase, Ca++ transporting, ubiquitous |
| 158 | M63962 | ATP4A | ATPase, H+/K+ exchanging, alpha polypeptide |
| 159 | M75110 | ATP4B | ATPase, H+/K+ exchanging, beta polypeptide |
| 160 | AA987754 | B3GALT4 | UDP-Gal: betaGlcNAc beta 1,3- |
| | | | galactosyltransferase, polypeptide 4 |
| 161 | U92715 | BCAR3 | breast cancer anti-estrogen resistance 3 |
| 162 | H09748 | BCL11B | B-cell lymphoma/leukaemia 11B |
| 163 | AA609134 | BIRC6 | Baculoviral IAP repeat-containing 6 |
| 164 | AA429149 | C11ORF9 | chromosome 11open reading frame 9 |
| 165 | AI186263 | C21ORF11 | chromosome 21 open reading frame 11 |
| 166 | AI290349 | C5 | complement component 5 |
| 167 | J03037 | CA2 | carbonic anhydrase II |
| 168 | AA687964 | CAMK2D | Calcium/calmodulin-dependent protein kinase |
| | | | (CaM kinase) II delta |
| 169 | U26710 | CBLB | Cas-Br-M (murine) ectropic retroviral |
| | | | transforming sequence b |
| 170 | M80462 | CD79A | CD79A antigen (immunoglobulin-associated |
| | | | alpha) |
| 171 | M83077 | CD80 | CD80 antigen |
| 172 | M80629 | CDC2L5 | cell division cycle 2-like 5 (cholinesterase- |
| | | | related cell division controller) |
| 173 | J03483 | CHGA | chromogranin A (parathyroid secretory protein |
| | | | 1) |
| 174 | U62431 | CHRNA2 | cholinergic receptor, nicotinic, alpha |
| | | | polypeptide 2 (neuronal) |
| 175 | J02883 | CLPS | colipase, pancreatic |
| 176 | AI208243 | CNOT3 | CCR4-NOT transcription complex, subunit 3 |
| 177 | M81379 | COL4A3 | collagen, type IV, alpha 3 (Goodpasture |
| | | | antigen) |
| 178 | X54412 | COL9A1 | collagen, type IX, alpha 1 |
| 179 | U19977 | CPA2 | carboxypeptidase A2 (pancreatic) |
| 180 | R45683 | CPD | Carboxypeptidase D |
| 181 | AI081228 | CrkRS | CDC2-related protein kinase 7 |
| 182 | AA992910 | CTXL | cortic al thymocyte receptor (X. laevis CTX) |
| | | | like |
| 183 | L16876 | CYP2C18 | cytochrome P-450 2C18 |
| 184 | J04813 | CYP3A5 | cytochrome P450, subfamily IIIA (niphedipine |
| | | | oxidase), polypeptide 5 |
| 185 | D00408 | CYP3A7 | cytochrome P450, subfamily IIIA, polypeptide 7 |
| 186 | AA921756 | DIA4 | diaphorase (NADH/NADPH) (cytochrome b-5 |
| | | | reductase) |
| 187 | R37098 | DKFZp547M236 | hypothetical protein DKFZp547M236 |
| 188 | AI306435 | DKFZP586A0522 | DKFZP586A0522 protein |
| 189 | U36341 | DXS1357E | accessory proteins BAP31/BAP29 |
| 190 | AA868848 | ELSPBP1 | epididymal sperm binding protein 1 |
| 191 | U91510 | ENTPD2 | ectonucleoside triphosphate |
| | | | diphosphohydrolase 2 |
| 192 | D16305 | ERCC5 | excision repair cross-complementing rodent |
| | | | repair deficiency, complementation group 5 |
| | | | (xeroderma pigmentosum, complementation |
| | | | group G (Cockayne syndrome)) |
| 193 | M10617 | FABP1 | fatty acid binding protein 1, liver |
| 194 | L10320 | FBP1 | fructose, 6-bisphosphatase 1 |
| 195 | AA573905 | FCGBP | Fc fragment of IgG binding protein |
| 196 | AA678103 | FKBP5 | FK506-binding protein 5 |
| 197 | AI096444 | FLJ10707 | hypothetical protein FLJ10707 |
| 198 | AA650356 | FLJ10826 | hypothetical protein FLJ10826 |
| 199 | AA137133 | FLJ20043 | hypothetical protein FLJ20043 |
| 200 | AA825438 | FLJ20154 | hypothetical protein FLJ20154 |
| 201 | AA112198 | FLJ20296 | hypothetical protein FLJ20296 |
| 202 | N79769 | FLJ20331 | hypothetical protein FLJ20331 |
| 203 | AA844597 | FLJ22174 | hypothetical protein FLJ22174 |
| 204 | U30461 | GABRA4 | gamma-aminobutyric acid (GABA) A receptor, |
| | | | alpha 4 |
| 205 | Z48475 | GCKR | glucokinase (hexokinase 4) regulatory protein |
| 206 | U10550 | GEM | GTP-binding protein overexpressed in skeletal |
| | | | muscle |
| 207 | M63154 | GIF | gastric intrinsic factor (vitamin B synthesis) |
| 208 | AA523541 | GILZ | glucocorticoid-induced leucine zipper |
| 209 | M37400 | GOT1 | glutamic-oxaloacetic transaminase 1, soluble |
| | | | (aspartate aminotransferase 1) |
| 210 | U11287 | GRIN2B | glutamate receptor, ionotropic, N-methyl D- |
| | | | aspartate 2B |
| 211 | AA993251 | GSTA2 | glutathione S-transferase A2 |
| 212 | L13275 | GSTA3 | glutathione S-transferase A3 |
| 213 | R12013 | HDCMC04P | hypothetical protein HDCMC04P |
| 214 | J04178 | HEXA | Human abnormal beta-hexosaminidase alpha |
| | | | chain (HEXA) mRNA, partial cds |
| 215 | AI088680 | HIP-55 | src homology 3 domain-containing protein HIP- |
| | | | 55 |
| 216 | M75126 | HK1 | hexokinase 1 |
| 217 | X83618 | HMGCS2 | 3-hydroxy-3-methylglutaryl-Coenzyme A |
| | | | synthase 2 (mitochondrial) |
| 218 | AF040714 | HOXA10 | homeo box A10 |
| 219 | AA101819 | HOXC13 | homeo box C13 |
| 220 | L76465 | HPGD | hydroxyprostaglandin dehydrogenase 15- |
| | | | (NAD) |
| 221 | U24186 | HSU24186 | replication protein A complex 34 kd subunit |
| | | | homolog Rpa4 |
| 222 | U52521 | HSU52521 | arfaptin 1 |
| 223 | AF049524 | HYPA | Huntingtin-interacting protein A |
| 224 | X67292 | IGHM | immunoglobulin heavy constant mu |
| 225 | X59770 | IL1R2 | interleukin 1 receptor, type II |
| 226 | U61263 | ILVBL | ilvB (bacterial acetolactate synthase)-like |
| 227 | X16260 | ITIH1 | inter-alpha (globulin) inhibitor, H1 polypeptide |
| 228 | W76477 | JUN | v-jun avian sarcoma virus 17 oncogene |
| | | | homolog |
| 229 | M64676 | KCNC4 | potassium voltage-gated channel, Shaw-related |
| | | | subfamily, member 4 |
| 230 | AA845511 | KCNJ16 | potassium inwardly-rectifying channel, |
| | | | subfamily J, member 16 |
| 231 | U33632 | KCNK1 | potassium channel, subfamily K, member 1 |
| | | | (TWIK) |
| 232 | R98339 | KIAA0105 | Wilms' tumour 1-associating protein |
| 233 | AB007859 | KIAA0399 | KIAA0399 protein |
| 234 | AF007170 | KIAA0452 | DEME-6 protein |
| 235 | AB014578 | KIAA0678 | KIAA0678 protein |
| 236 | H49431 | KIAA0720 | KIAA0720 protein |
| 237 | H15919 | KIAA0725 | KIAA0725 protein |
| 238 | AA489065 | KIAA0744 | histone deacetylase 7B |
| 239 | AA676319 | KIAA0865 | KIAA0865 protein |
| 240 | AA443202 | KIAA1053 | KIAA1053 protein |
| 241 | N54300 | KIAA1500 | KIAA1500 protein |
| 242 | M59964 | KITLG | KIT ligand |
| 243 | X73502 | KRT20 | cytokeratin 20 |
| 244 | X67683 | KRT4 | keratin K4a |
| 245 | M87842 | LGALS2 | lectin, galactoside-binding, soluble, 2 (galectin |
| | | | 2) |
| 246 | D26309 | LIMK1 | LIM domain kinase 1 |
| 247 | U24576 | LMO4 | LIM domain only 4 |
| 248 | AA458747 | LOC51092 | CGI-40 protein |
| 249 | H25172 | LOC51247 | hypothetical protein |
| 250 | AA503989 | LOC51635 | CGI-86 protein |
| 251 | AI093595 | LOC55895 | 22 kDa peroxisomal membrane protein-like |
| 252 | AA363794 | LOC55914 | erbb2-interacting protein ERBIN |
| 253 | AA524740 | LOC56928 | hypothetical protein from EUROIMAGE 42353 |
| 254 | D50678 | LRP8 | low density lipoprotein receptor-related protein |
| | | | 8, apolipoprotein e receptor |
| 255 | AA434024 | LSS | lanosterol synthase (2,3-oxidosqualene- |
| | | | lanosterol cyclase) |
| 256 | M83202 | LTF | lactotransferrin |
| 257 | AA609685 | M11S1 | membrane component, chromosome 11, surface |
| | | | marker 1 |
| 258 | U93163 | MAGEB1 | melanoma antigen, family B, 1 |
| 259 | M15800 | MAL | mal, T-cell differentiation protein |
| 260 | X98400 | MASP2 | mannan-binding lectin serine protease 2 |
| 261 | X00371 | MB | myoglobin |
| 262 | M62397 | MCC | mutated in colorectal cancers |
| 263 | AB011144 | MCM3AP | minichromosome maintenance deficient (S. cerevisiae) |
| | | | 3-associated protein |
| 264 | U49020 | MEF2A | MADS box transcription enhancer factor 2, |
| | | | polypeptide A (myocyte enhancer factor 2A) |
| 265 | AF017418 | MEIS2 | Meis (mouse) homolog 2 |
| 266 | X92841 | MICA | MHC class I polypeptide-related sequence A |
| 267 | AA813616 | MID2 | midline 2 |
| 268 | U02478 | MLLT4 | myeloid/lymphoid or mixed-lineage leukemia |
| | | | (trithorax (Drosophila) homolog); translocated |
| | | | to, 4 |
| 269 | N70019 | MT1E | metallothionein 1E (functional) |
| 270 | D20201 | MT1L | metallothionein 1L |
| 271 | AI094778 | MT2A | metallothionein 2A |
| 272 | X79882 | MVP | major vault protein |
| 273 | AA704060 | NDUFS1 | NADH dehydrogenase (ubiquinone) Fe—S |
| | | | protein 1 (75 kD) (NADH-coenzyme Q |
| | | | reductase) |
| 274 | AA340728 | NR2F2 | nuclear receptor subfamily 2, group F, member 2 |
| 275 | M23204 | OAT | ornithine aminotransferase (gyrate atrophy) |
| 276 | L24804 | P23 | unactive progesterone receptor, 23 kD |
| 277 | L15533 | PAP | pancreatitis-associated protein |
| 278 | L25597 | PAX2 | paired box gene 2 |
| 279 | U57317 | PCAF | p300/CBP-associated factor |
| 280 | C05229 | PDK4 | pyruvate dehydrogenase kinase, isoenzyme 4 |
| 281 | AF012281 | PDZK1 | PDZ domain containing 1 |
| 282 | J00287 | PGA3 | pepsinogen 3, group I (pepsinogen A) |
| 283 | M23077 | PGC | pepsinogen C |
| 284 | U79280 | PIPPIN | ortholog of rat pippin |
| 285 | U59305 | PK428 | Ser-Thr protein kinase related to the myotonic |
| | | | dystrophy protein kinase |
| 286 | AA234962 | PKP3 | plakophilin 3 |
| 287 | D87810 | PMM1 | phosphomannomutase 1 |
| 288 | S74349 | PPARA | peroxisome proliferative activated receptor, |
| | | | alpha |
| 289 | AF034803 | PPFIBP2 | PTPRF interacting protein, binding protein 2 |
| | | | (liprin beta 2) |
| 290 | X80910 | PPP1CB | protein phosphatase 1, catalytic subunit, beta |
| | | | isoform |
| 291 | L42373 | PPP2R5A | protein phosphatase 2, regulatory subunit B |
| | | | (B56), alpha isoform |
| 292 | H67736 | PPY2 | pancreatic polypeptide 2 |
| 293 | R26785 | PRSS8 | protease, serine, 8 (prostasin) |
| 294 | AF043498 | PSCA | prostate stem cell antigen |
| 295 | U57094 | RAB27A | RAB27A, member RAS oncogene family |
| 296 | AA312113 | RBL1 | retinoblastoma-like 1 (p107) |
| 297 | AA531163 | REC14 | Recombination protein REC14 |
| 298 | M18963 | REG1A | regenerating islet-derived 1 alpha (pancreatic |
| | | | stone protein, pancreatic thread protein) |
| 299 | L08010 | REG1B | regenerating islet-derived 1 beta (pancreatic |
| | | | stone protein, pancreatic thread protein) |
| 300 | Y12812 | RFXAP | regulatory factor X-associated protein |
| 301 | Y17108 | RHBDL | rhomboid (veinlet, Drosophila)-like |
| 302 | T95199 | RNAHP | RNA helicase-related protein |
| 303 | AA778308 | RNASE1 | ribonuclease, RNase A family, 1 (pancreatic) |
| 304 | AI241742 | RPL36 | Ribosomal protein L36 |
| 305 | AA308062 | S100P | S100 calcium-binding protein P |
| 306 | W42910 | SEC22C | vesicle trafficking protein |
| 307 | H07129 | SEC24D | SEC24 (S. cerevisiae) related gene family, |
| | | | member D |
| 308 | AI275118 | SENP7 | Sentrin/SUMO-specific protease |
| 309 | L13470 | SERPINA7 | serine (or cysteine) proteinase inhibitor, clade A |
| | | | (alpha antiproteinase, antitrypsin), member 7 |
| 310 | AA873052 | SERPINI1 | serine (or cysteine) proteinase inhibitor, clade I |
| | | | (neuroserpin), member 1 |
| 311 | Y10032 | SGK | serum/glucocorticoid regulated kinase |
| 312 | AI090954 | SH3BGRL2 | SH3 domain binding glutamic acid-rich protein |
| | | | like 2 |
| 313 | X15218 | SKI | v-ski avian sarcoma viral oncogene homolog |
| 314 | AB007448 | SLC22A4 | solute carrier family 22 (organic cation |
| | | | transporter), member 4 |
| 315 | U25147 | SLC25A1 | solute carrier family 25 (mitochondrial carrier; |
| | | | citrate transporter), member 1 |
| 316 | AA521247 | SLC25A20 | Solute carrier family 25 (carnitine/acylcarnitine |
| | | | translocase), member 20 |
| 317 | J02966 | SLC25A4 | solute carrier family 25 (mitochondrial carrier; |
| | | | adenine nucleotide translocator), member 4 |
| 318 | AA621201 | SLC30A3 | solute carrier family 30 (zinc transporter), |
| | | | member 3 |
| 319 | AA446144 | SLC7A8 | solute carrier family 7 (cationic amino acid |
| | | | transporter, y+ system), member 8 |
| 320 | M96067 | SLC9A1 | solute carrier family 9 (sodium/hydrogen |
| | | | exchanger), isoform 1 (antiporter, Na+/H+, |
| | | | amiloride sensitive) |
| 321 | AF068180 | SLP65 | B cell linker protein |
| 322 | AI125978 | SNX2 | sorting nexin 2 |
| 323 | L07335 | SOX2 | SRY (sex determining region Y)-box 2 |
| 324 | L14865 | SSTR5 | somatostatin receptor 5 |
| 325 | S45936 | ST5 | suppression of tumorigenicity 5 |
| 326 | AA683542 | STAU2 | staufen (Drosophila, RNA-binding protein) |
| | | | homolog 2 |
| 327 | AA522445 | SYTL2 | Synaptotagmin-like 2 |
| 328 | AA443786 | SYTL2 | Synaptotagmin-like 2 |
| 329 | AA614579 | TFF1 | trefoil factor 1 (breast cancer, estrogen- |
| | | | inducible sequence expressed in) |
| 330 | AA741431 | TFF2 | trefoil factor 2 (spasmolytic protein 1) |
| 331 | M19713 | TPM1 | tropomyosin 1 (alpha) |
| 332 | M12125 | TPM2 | tropomyosin 2 (beta) |
| 333 | X01410 | TRB@ | T cell receptor beta locus |
| 334 | AI300188 | UBE1 | ubiquitin-activating enzyme E1 (A1S9T and |
| | | | BN75 temperature sensitivity complementing) |
| 335 | AA774430 | UBL3 | ubiquitin-like 3 |
| 336 | M57899 | UGT1A1 | UDP glycosyltransferase 1 family, polypeptide |
| | | | A1 |
| 337 | W22795 | USP11 | ubiquitin specific protease 11 |
| 338 | AF000994 | UTY | ubiquitously transcribed tetratricopeptide repeat |
| | | | gene, Y chromosome |
| 339 | D88154 | VILL | villin-like |
| 340 | Z19002 | ZNF145 | zinc finger protein 145 (Kruppel-like, expressed |
| | | | in promyelocytic leukemia) |
| 341 | X78931 | ZNF272 | zinc finger protein 272 |
| 342 | N29536 | | ESTs |
| 343 | AA528190 | | ESTs |
| 344 | AI025000 | | ESTs, Weakly similar to PI-3 kinase |
| | | | [H. sapiens] |
| 345 | AI299327 | | ESTs |
| 346 | AI271678 | | ESTs |
| 347 | AA419568 | | ESTs |
| 348 | AA628346 | | EST |
| 349 | AA479350 | | ESTs |
| 350 | AA991482 | | Human DNA sequence from clone RP1-304B14 |
| | | | on chromosome 6. Contains a gene for a novel |
| | | | protein and a part of a gene for a novel protein |
| | | | with two isoforms. |
| 351 | H89110 | | ESTs |
| 352 | AA860341 | | ESTs |
| 353 | AI275857 | | ESTs |
| 354 | H61936 | | ESTs |
| 355 | AI243456 | | ESTs |
| 356 | W37605 | | ESTs |
| 357 | AA669034 | | Homo sapiens cDNA: FLJ23125 fis, clone |
| | | | LNG08217 |
| 358 | AA187834 | | EST |
| 359 | AA019961 | | Homo sapiens cDNA: FLJ22811 fis, clone |
| | | | KAIA2944 |
| 360 | AA193416 | | ESTs, Weakly similar to AF064254 1 very |
| | | | long-chain acyl-CoA synthetase homolog 1 |
| | | | [H. sapiens] |
| 361 | AA604353 | | Homo sapiens mRNA; cDNA DKFZp564F2072 |
| | | | (from clone DKFZp564F2072) |
| 362 | AA599046 | | EST |
| 363 | AA150200 | | ESTs, Weakly similar to tuftelin [M. musculus] |
| 364 | AA481396 | | ESTs |
| 365 | AA147751 | | Homo sapiens cDNA FLJ14146 fis, clone |
| | | | MAMMA1002947 |
| 366 | AA907673 | | ESTs |
| 367 | W63676 | | ESTs |
| 368 | AA609467 | | EST |
| 369 | AI281337 | | ESTs |
| 370 | F09892 | | ESTs |
| 371 | AA814111 | | ESTs |
| 372 | AI366242 | | ESTs |
| 373 | N34387 | | EST |
| 374 | N24387 | | Homo sapiens cDNA FLJ10532 fis, clone |
| | | | NT2RP2001044 |
| 375 | AA913947 | | ESTs |
| 376 | W79248 | | ESTs |
| 377 | AA532999 | | ESTs, Weakly similar to/prediction |
| 378 | X90579 | | H. sapiens DNA for cyp related pseudogene |
| 379 | AA063157 | | ESTs |
| 380 | F21002 | | ESTs |
| 381 | R39044 | | Homo sapiens clone 25194 mRNA sequence |
| 382 | H10766 | | ESTs, Weakly similar to dJ1170K4.1 |
| | | | [H. sapiens] |
| 383 | AA847242 | | ESTs, Weakly similar to G786_HUMAN |
| | | | PROTEIN GS3786 [H. sapiens] |
| 384 | AI248721 | | ESTs |
| 385 | AA291066 | | ESTs |
| 386 | AA429441 | | ESTs |
| 387 | AA421326 | | Homo sapiens cDNA: FLJ21918 fis, clone |
| | | | HEP04006 |
| 388 | AI248610 | | ESTs |
| 389 | AI056871 | | ESTs |
| 390 | AA489368 | | ESTs |
| 391 | AI122561 | | Homo sapiens cDNA: FLJ22603 fis, clone |
| | | | HSI04564 |
| 392 | AA600238 | | ESTs |
| 393 | D62524 | | ESTs |
| 394 | AA806114 | | ESTs |
| 395 | AA680050 | | ESTs |
| 396 | AA430571 | | ESTs |
| 397 | AA197086 | | ESTs |
| 398 | N50517 | | ESTs |
| 399 | AI280964 | | Homo sapiens cDNA: FLJ22055 fis, clone |
| | | | HEP09645 |
| 400 | AA019195 | | ESTs |
| 401 | AA905751 | | ESTs |
| 402 | AA602976 | | ESTs |
| 403 | N48008 | | EST |
| 404 | AA480873 | | ESTs |
| 405 | T80844 | | ESTs |
| 406 | AI088309 | | ESTs |
| 407 | T65992 | | ESTs |
| 408 | AF058075 | | Homo sapiens clone ASPBLL54 |
| | | | immunoglobulin lambda light chain VJ region |
| | | | mRNA, partial cds |
| 409 | AA291355 | | Homo sapiens cDNA: FLJ22253 fis, clone |
| | | | HRC02763 |
| 410 | T24065 | | ESTs |
| 411 | AI076929 | | ESTs, Weakly similar to Homolog of rat |
| | | | Zymogen granule membrane protein |
| | | | [H. sapiens] |
| 412 | AA621983 | | Homo sapiens cDNA: FLJ22495 fis, clone |
| | | | HRC11205, highly similar to HSA223366 |
| | | | Homo sapiens mRNA for OCIM (Oncogene in |
| | | | Multiple Myeloma) protein |
| 413 | N79592 | | ESTs |
| 414 | AA157981 | | ESTs |
| 415 | R44001 | | ESTs |
| 416 | AA179812 | | Homo sapiens cDNA: FLJ21918 fis, clone |
| | | | HEP04006 |
| 417 | AA579711 | | Homo sapiens cDNA: FLJ23306 fis, clone |
| | | | HEP11541 |
| 418 | T91207 | | ESTs |
| 419 | Z40838 | | Thyroid hormone receptor interactor 3 |
| 420 | AI089525 | | ESTs |
| 421 | R39856 | | Human DNA sequence from clone RP5-995J12 |
| | | | on chromosome 20q12 Contains part of a gene |
| | | | similar to ganglioside-induced differentiation |
| | | | associated protein 1 |
| 422 | AA433914 | | ESTs |
| 423 | AA743462 | | ESTs |
| 424 | R69133 | | ESTs |
| 425 | AA002191 | | ESTs |
| 426 | AA977256 | | ESTs |
| 427 | AA781175 | | ESTs |
| 428 | AA402013 | | ESTs |
| 429 | AA701871 | | EST |
| 430 | AA625854 | | EST |
| 431 | AA442883 | | ESTs |
| 432 | AA682425 | | ESTs |
| 433 | T24091 | | EST |
| 434 | AA968696 | | ESTs |
| 435 | AA747289 | | ESTs |
| 436 | AI279221 | | ESTs |
| 437 | AA628328 | | ESTs |
| 438 | AA351680 | | ESTs |
| 439 | AA688275 | | ESTs |
| 440 | H81716 | | ESTs |
| 441 | AA758321 | | ESTs |
| 442 | AA279460 | | Homo sapiens mRNA; cDNA DKFZp564N196 |
| | | | (from clone DKFZp564N196) |
| 443 | N22132 | | Homo sapiens cDNA: FLJ21841 fis, clone |
| | | | HEP01831 |
| 444 | AA985007 | | Homo sapiens mRNA; cDNA DKFZp564A026 |
| | | | (from clone DKFZp564A026) |
| 445 | H94248 | | ESTs |
| 446 | AA804409 | | ESTs |
| 447 | L44436 | | ESTs |
| 448 | AA478951 | | EST |
| 449 | AA451866 | | ESTs |
| 450 | H04150 | | ESTs |
| 451 | AA148523 | | Homo sapiens cDNA: FLJ21032 fis, clone |
| | | | CAE07365 |
| 452 | AA490225 | | ESTs |
| 453 | H70955 | | ESTs |
| 454 | R54643 | | ESTs |
| 455 | AA614273 | | ESTs, Weakly similar to CPT1_HUMAN |
| | | | CARNITINE O- |
| | | | PALMITOYLTRANSFERASE I, |
| | | | MITOCHONDRIAL LIVER ISOFORM |
| | | | [H. sapiens] |
| 456 | AI125859 | | ESTs |
| 457 | R44423 | | ESTs, Weakly similar to KIAA0927 protein |
| | | | [H. sapiens] |
| 458 | AI312787 | | ESTs |
| 459 | AA488881 | | EST |
| 460 | AA521097 | | Likely ortholog of mouse coiled coil forming |
| | | | protein 1 |
| 461 | AA598844 | | ESTs, Weakly similar to Attractin [H. sapiens] |
| 462 | R46597 | | Homo sapiens mRNA; cDNA DKFZp434P1018 |
| | | | (from clone DKFZp434P1018); partial cds |
| 463 | AA292973 | | ESTs |

### INDUSTRIAL APPLICABILITY

The gene-expression analysis of DGC described herein, obtained through a combination of laser-capture dissection and genome-wide cDNA microarray, has identified specific genes as targets for cancer prevention and therapy. Based on the expression of a subset of these differentially expressed genes, the present invention provides a molecular diagnostic markers for identifying or detecting DGC.

The methods described herein are also useful in the identification of additional molecular targets for prevention, diagnosis and treatment of DGC. The data reported herein add to a comprehensive understanding of DGC, facilitate development of novel diagnostic strategies, and provide clues for identification of molecular targets for therapeutic drugs and preventative agents. Such information contributes to a more profound understanding of gastric tumorigenesis, and provide indicators for developing novel strategies for diagnosis, treatment, and ultimately prevention of DGC.

All patents, patent applications, and publications cited herein are incorporated by reference in their entirety. Furthermore, while the invention has been described in detail and with reference to specific embodiments thereof, it will be apparent to one skilled in the art that various changes and modifications can be made therein without departing from the spirit and scope of the invention.

### REFERENCES

1. Parkin, D. M., Pisani, P., Ferlay, J. Estimates of the worldwide incidence of 25 major cancers in 1990. Int. J. Cancer, 80: 827-841, 1999.
2. Lauren, P. The two histological main types of gastric carcinoma: diffuse and so-called intestinal-type carcinoma Acta Path. Microbiol. Scand., 64: 31-49, 1965.
3. Correa, P., Chen, V. W. Gastric cancer. Cancer Surv., 19-20: 55-76, 1994.
4. Ming, S. C., review article: Cellular and molecular pathology of gastric carcinoma and precursor lesions: A critical review. Gastric Cancer, 1: 31-50, 1998.
5. Hesketh, R. The Oncogene and Tumour Suppressor Gene Facts Book. San Diego: Academic Press, 1997.
6. Werner, M., Becker, K. F., Keller, G., Hofler, H. Gastric adenocarcinoma: pathomorphology and molecular pathology. J. Cancer Res. Clin. Oncol., 127: 207-216, 2001.
7. Guilford, P., Hopkins, J., Harraway, J., McLeod, M., McLeod, N., Harawira, P., Taite, H., Scoular, R., Miller, A, Reeve, A E. E-cadherin germline mutations in familial gastric cancer. Nature(Lond.), 392: 402-405, 1998.
8. Kitahara, O., Furukawa, Y., Tanaka, T., Kihara, C., Ono, K., Yanagawa, R., Nita, M. E., Takagi, T., Nakamura, Y., Tsunoda, T. Alterations of gene expression during colorectal carcinogenesis revealed by cDNA microarrays after laser-capture microdissection of tumor tissues and normal epithelia Cancer Res., 61: 3544-3549, 2001.
9. Golub, T. R., Slonim, D. K., Tamayo, P., Huard, C., Gaasenbeek, M., Mesirov, J. P., Coller, H., Loh, M. L., Downing, J. R., Caligiuri, M. A., Bloomfield, C. D., Lander, E. S. Molecular classification of cancer: class discovery and class prediction by gene expression monitoring. Science, 286: 531-537, 1999.
10. Yanagawa, R., Furukawa, Y., Tsunoda, T., Kitahara, O., Kameyama, M., Murata, K., Ishikawa, O., Nakamura, Y. Genome-wide screening of genes showing altered expression in liver metastases of human colorectal cancers by cDNA microarray. Neoplasia, 3: 395-401, 2001.
11. Hippo, Y., Yashiro, M., Ishii, M., Taniguchi, H., Tsutsumi, S., Hirakawa, K., Kodama, T., Aburatani, H. Differential gene expression profiles of scirrhous gastric cancer cells with high metastatic potential to peritoneum or lymph nodes. Cancer Res., 61: 889-895, 2001.
12. El-Rifai, W., Frierson, H. F. Jr., Harper, J. C., Powell, S. M., Knuutila, S. Expression profiling of gastric adenocarcinoma using cDNA array. Int. J. Cancer, 92: 832-838, 2001
13. Molina M A, Codony-Servat J, Albanell J, Rojo F, Arribas J and Baselga J: Trastuzumab (herceptin), a humanized anti-Her2 receptor monoclonal antibody, inhibits basal and activated Her2 ectodomain cleavage in breast cancer cells. Cancer Res 61: 4744-9. 2001.
14. O'Dwyer M E and Druker B J: Status of bcr-abl tyrosine kinase inhibitors in chronic myelogenous leukemia. Curr Opin Oncol 12: 594-7, 2000

1. A method of diagnosing DGC or a predisposition to developing DGC in a subject, comprising determining a level of expression of a DGC-associated gene in a patient derived biological sample, wherein an increase or decrease of said level compared to a normal control level of said gene indicates that said subject suffers from or is at risk of developing DGC.

2. The method of claim 1, wherein said DGC-associated gene is selected from the group consisting of DGC 1-136, wherein an increase in said level compared to a normal control level indicates said subject suffers from or is at risk of developing DGC.

3. The method of claim 2, wherein said increase is at least 10% greater than said normal control level.

4. The method of claim 1, wherein said DGC-associated gene is selected from the group consisting of DGC 137-463, wherein a decrease in said level compared to a normal control level indicates said subject suffers from or is at risk of developing DGC.

5. The method of claim 4, wherein said decrease is at least 10% lower than said normal control level.

6. The method of claim 1, wherein said method further comprises determining said level of expression of a plurality of DGC-associated genes.

7. The method of claim 1, wherein the expression level is determined by any one method select from group consisting of:
(a) detecting the mRNA of the DGC-associated genes,
(b) detecting the protein encoded by the DGC-associated genes, and
(c) detecting the biological activity of the protein encoded by the DGC-associated genes.

8. The method of claim 7, wherein said detection is carried out on a DNA array.

9. The method of claim 1, wherein said biological sample comprises a mucosal cell.

10. The method of claim 1, wherein said biological sample comprises a tumor cell.

11. The method of claim 1, wherein said biological sample comprises a gastric cancer cell.

12. A DGC reference expression profile, comprising a pattern of gene expression of two or more genes selected from the group consisting of DGC 1-463.

13. A DGC reference expression profile, comprising a pattern of gene expression of two or more genes selected from the group consisting of DGC 1-136.

14. A DGC reference expression profile, comprising a pattern of gene expression of two or more genes selected from the group consisting of DGC 137-463.

15. A method of screening for a compound for treating or preventing DGC, said method comprising the steps of:
a) contacting a test compound with a polypeptide encoded by DGC 1-463;
b) detecting the binding activity between the polypeptide and the test compound; and
c) selecting a compound that binds to the polypeptide.

16. A method of screening for a compound for treating or preventing DGC, said method comprising the steps of:
a) contacting a candidate compound with a cell expressing one or more marker genes, wherein the one or more marker genes is selected from the group consisting of DGC 1-463; and
b) selecting a compound that reduces the expression level of one or more marker genes selected from the group consisting of DGC 1-136, or elevates the expression level of one or more marker genes selected from the group consisting of DGC 137-463.

17. A method of screening for a compound for treating or preventing DGC, said method comprising the steps of:
a) contacting a test compound with a polypeptide encoded by selected from the group consisting of DGC 1-463;
b) detecting the biological activity of the polypeptide of step (a); and
c) selecting a compound that suppresses the biological activity of the polypeptide encoded by DGC 1-136 in comparison with the biological activity detected in the absence of the test compound, or enhances the biological activity of the polypeptide encoded by DGC 137-463 in comparison with the biological activity detected in the absence of the test compound.

18. The method of claim 16, wherein said test cell comprises a gastric cancer cell.

19. A method of screening for compound for treating or preventing DGC, said method comprising the steps of:
a) contacting a candidate compound with a cell into which a vector comprising the transcriptional regulatory region of one or more marker genes and a reporter gene that is expressed under the control of the transcriptional regulatory region has been introduced, wherein the one or more marker genes are selected from the group consisting of DGC 1-463
b) measuring the activity of said reporter gene; and
c) selecting a compound that reduces the expression level of said reporter gene when said marker gene is an up-regulated marker gene selected from the group consisting of DGC 1-136 or that enhances the expression level of said reporter gene when said marker gene is a down-regulated marker gene selected from the group consisting of DGC 137-463, as compared to a control.

20. A kit comprising a detection reagent which binds to two or more nucleic acid sequences selected from the group consisting of DGC 1-463.

21. An array comprising a nucleic acid which binds to two or more nucleic acid sequences selected from the group consisting of DGC 1-463.

22. A method of treating or preventing DGC in a subject comprising administering to said subject an antisense composition, said composition comprising a nucleotide sequence complementary to a coding sequence selected from the group consisting of DGC 1-136.

23. A method of treating or preventing DGC in a subject comprising administering to said subject a siRNA composition, wherein said composition reduces the expression of a nucleic acid sequence selected from the group consisting of DGC 1-136.

24. A method for treating or preventing DGC in a subject comprising the step of administering to said subject a pharmaceutically effective amount of an antibody or fragment thereof that binds to a protein encoded by any one gene selected from the group consisting of DGC 1-136.

25. A method of treating or preventing DGC in a subject comprising administering to said subject a vaccine comprising a polypeptide encoded by a nucleic acid selected s from the group consisting of DGC 1-136 or an immunologically active fragment of said polypeptide, or a polynucleotide encoding the polypeptide.

26. A method of treating or preventing DGC in a subject comprising administering to said subject a compound that increases the expression or activity of DGC 137-463.

27. A method for treating or preventing DGC in a subject, said method comprising the step of administering a compound that is obtained by the method according to any one of claims 15-19.

28. A method of treating or preventing DGC in a subject comprising administering to said subject a pharmaceutically effective amount of polynucleotide select from group consisting of DGC 137-463, or polypeptide encoded by thereof.

29. A composition for treating or preventing DGC, said composition comprising a pharmaceutically effective amount of an antisense polynucleotide or small interfering RNA against a polynucleotide select from group consisting of DGC 1-136 as an active ingredient, and a pharmaceutically acceptable carrier.

30. A composition for treating or preventing DGC, said composition comprising a pharmaceutically effective amount of an antibody or fragment thereof that binds to a protein encoded by any one gene selected from the group consisting of DGC 1-136 as an active ingredient, and a pharmaceutically acceptable carrier.

31. A composition for treating or preventing DGC, said composition comprising a pharmaceutically effective amount of the compound selected by the method of any one of claims 15-19 as an active ingredient, and a pharmaceutically acceptable carrier.


SEQUENCE LISTING (12 sequences)

SEQ ID NO: 1 - DNA, 23 bp, Artificial Sequence
  An artificially synthesized primer sequence for RT-PCR
tgtgtggctgggacctttaggaa

SEQ ID NO: 2 - DNA, 25 bp, Artificial Sequence
  An artificially synthesized primer sequence for RT-PCR
gaatcatactaggaccgatcttact

SEQ ID NO: 3 - DNA, 23 bp, Artificial Sequence
  An artificially synthesized primer sequence for RT-PCR
tccctggaaaaggagcttcagta

SEQ ID NO: 4 - DNA, 23 bp, Artificial Sequence
  An artificially synthesized primer sequence for RT-PCR
gataacactgtctcggtaccaca

SEQ ID NO: 5 - DNA, 24 bp, Artificial Sequence
  An artificially synthesized primer sequence for RT-PCR
caagagtgagatgtagaaagttgt

SEQ ID NO: 6 - DNA, 23 bp, Artificial Sequence
  An artificially synthesized primer sequence for RT-PCR
gtttgagagtggtactacacttc

SEQ ID NO: 7 - DNA, 25 bp, Artificial Sequence
  An artificially synthesized primer sequence for RT-PCR
agacgcatgttatggtgctaatgta

SEQ ID NO: 8 - DNA, 25 bp, Artificial Sequence
  An artificially synthesized primer sequence for RT-PCR
cattcgaacatacaccaacaactag

SEQ ID NO: 9 - DNA, 21 bp, Artificial Sequence
  An artificially synthesized primer sequence for RT-PCR
aacggctacctggtcctagac

SEQ ID NO: 10 - DNA, 22 bp, Artificial Sequence
  An artificially synthesized primer sequence for RT-PCR
tctcttgagcgcgagtcatttg

SEQ ID NO: 11 - DNA, 20 bp, Artificial Sequence
  An artificially synthesized primer sequence for RT-PCR
tttaacgctggtgggcagca

SEQ ID NO: 12 - DNA, 22 bp, Artificial Sequence
  An artificially synthesized primer sequence for RT-PCR
gaaaccctacccaagacaaata